(19) United States
(12) Patent Application Publication
(10) Pub. No.: US 2025/0361258 A1
(43) Pub. Date: Nov. 27, 2025

(54) PROCESSES FOR MAKING PRMT5 INHIBITORS

(71) Applicant: PRELUDE THERAPEUTICS INCORPORATED, Wilmington, DE (US)

(72) Inventors: Ganfeng CAO, Chadds Ford, PA (US); Andrew COMBS, Kennett Square, PA (US); Hongwu YU, Newark, DE (US); Peng WEI, Hockessin, DE (US)

(21) Appl. No.: 18/874,839

(22) PCT Filed: Jun. 14, 2023
(86) PCT No.: PCT/US2023/068408

§ 371 Date: Dec. 13, 2024

Related U.S. Application Data
(60) Provisional application No. 63/366,335, filed on Jun. 14, 2022.

(51) Int. Cl. C07H 17/06; A61K 31/7064; C07H 19/14

(52) U.S. Cl. CPC C07H 17/06; A61K 31/7064; C07H 19/14

(57) ABSTRACT

The disclosure provides processes for preparing the compound of formula (VIa-1) and pharmaceutically acceptable salts thereof. Intermediates useful in preparing the compound of formula (VIa-1) are also provided.

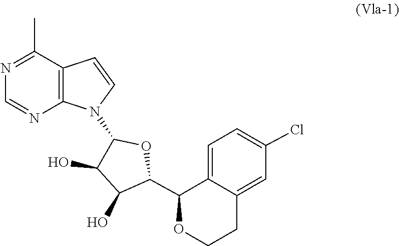 [SMILES: Cc1ncnc2c1ccn2[C@@H]1O[C@H]([C@@H]2OCCc3cc(Cl)ccc32)[C@@H](O)[C@H]1O]

### CROSS-REFERENCE TO RELATED APPLICATION

The present application claims priority to U.S. Patent Application No. 63/366,335, filed Jun. 14, 2022, the disclosure of which is incorporated by reference in its entirety.

### TECHNICAL FIELD

The disclosure is directed to methods of making PRMT5 inhibitors.

### BACKGROUND OF THE INVENTION

The compound of formula (VIa-1), or compound (VIa-1), (2S,3S,4R,5R)-2-((R)-6-chloroisochroman-1-yl)-5-(4-methyl-7H-pyrrolo[2,3-d]pyrimidin-7-yl)tetrahydrofuran-3,4-diol, is a PRMT5 inhibitor that is described in U.S. Pat. No. 10,711,007.

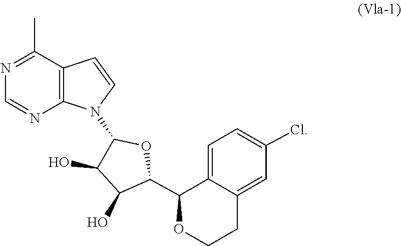 [SMILES: Cc1ncnc2c1ccn2[C@@H]1O[C@H]([C@@H]2OCCc3cc(Cl)ccc32)[C@@H](O)[C@H]1O]

A need exists for processes capable of preparing compound (VIa-1) and pharmaceutically acceptable salts thereof in high yields and with high stereochemical purity.

### SUMMARY OF THE INVENTION

The disclosure provides methods of preparing a compound of formula (VIa-1) and pharmaceutically acceptable salts thereof, and mixtures thereof, in high yields and with high stereochemical purity.

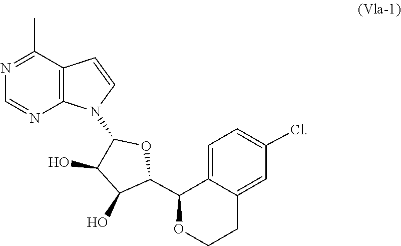 [SMILES: Cc1ncnc2c1ccn2[C@@H]1O[C@H]([C@@H]2OCCc3cc(Cl)ccc32)[C@@H](O)[C@H]1O]

### DETAILED DESCRIPTION OF ILLUSTRATIVE EMBODIMENTS

The disclosure may be more fully appreciated by reference to the following description, including the following definitions and examples. Certain features of the disclosed processes are described herein in the context of separate aspects, may also be provided in combination in a single aspect. Alternatively, various features of the disclosed processes that are, for brevity, described in the context of a single aspect, may also be provided separately or in any sub-combination.

In the present disclosure the singular forms “a,” “an,” and “the” include the plural reference, and reference to a particular numerical value includes at least that particular value, unless the context clearly indicates otherwise. Thus, for example, reference to “an organic solvent,” “organic solvent,” “an appropriate organic solvent,” and the like is a reference to one organic solvent or a mixture of organic solvents. When a range of values is expressed, another embodiment includes from the one particular and/or to the other particular value. All ranges are inclusive and combinable.

The modifier “about” should be considered as disclosing the range defined by the absolute values of the two endpoints. For example, the expression “from about 2 to about 4” also discloses the range “from 2 to 4.” When used to modify a single number, the term “about” refers to plus or minus 10% of the indicated number and includes the indicated number. For example, “about 10° C.” indicates a range of 9° C. to 11° C., and “about 1” means from 0.9-1.1.

“Pharmaceutically acceptable salt” refers to a salt of a compound of the disclosure that is pharmaceutically acceptable and that possesses the desired pharmacological activity of the parent compound. In particular, such salts are non-toxic and may be inorganic or organic acid addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methane-sulfonic acid, ethanesulfonic acid, 1,2-ethane-disulfonic acid, 2-hydroxyethane-sulfonic acid, benzenesulfonic acid, 4-chlorobenzenesulfonic acid, 2-naphthalenesulfonic acid, 4-toluene-sulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

The term “heteroaryl” when used alone or as part of a substituent group refers to a mono- or bicyclic-aromatic ring structure including carbon atoms as well as up to five heteroatoms selected from nitrogen, oxygen, and sulfur. Heteroaryl rings can include a total of 5, 6, 7, 8, 9, or 10 ring atoms. The term —C₅-C₁₀ heteroaryl refers to a heteroaryl group containing five to ten ring atoms. Examples of heteroaryl groups include but are not limited to, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like. Heteroaryl groups of the disclosure can be unsubstituted or substituted. In those embodiments wherein the heteroaryl group is substituted, the heteroaryl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “aryl” when used alone or as part of a substituent group refers to a mono- or bicyclic-aromatic carbon ring structure. Aryl rings can include a total of 5, 6, 7, 8, 9, or 10 ring atoms. Examples of aryl groups include but are not limited to, phenyl, napthyl, and the like. Aryl groups of the disclosure can be unsubstituted or substituted. In those embodiments wherein the aryl group is substituted, the aryl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy. Additional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

The term “heterocycloalkyl” when used alone or as part of a substituent group refers to any three to ten membered monocyclic or bicyclic, saturated ring structure containing at least one heteroatom selected from the group consisting of O, N and S. Heterocycloalkyl groups of the disclosure include monocyclic groups, as well as multicyclic groups such as bicyclic and tricyclic groups. In those embodiments having at least one multicyclic hetero-cycloalkyl group, the cyclic groups can share one common atom (i.e., spirocyclic). In other embodiments having at least one multicyclic heterocycloalkyl group, the cyclic groups share two common atoms. The term —C₃-C₆ heterocycloalkyl refers to a heterocycloalkyl group having between three and six carbon ring atoms. The term —C₃-C₁₀ heterocycloalkyl refers to a heterocycloalkyl group having between three and 10 rin atoms. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, azepanyl, diazepanyl, oxepanyl, dioxepanyl, azocanyl diazocanyl, oxocanyl, dioxocanyl, azaspiro[2.2]pentanyl, oxaazaspiro[3.3]heptanyl, oxaspiro[3.3]heptanyl, dioxaspiro[3.3]heptanyl, and the like. Heteroycloalkyl groups of the disclosure can be unsubstituted or substituted. In those embodiments wherein the heterocycloalkyl group is substituted, the heterocycloalkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy. Additional optional substitutents include —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂.

In some aspects, the disclosure is directed to processes for preparing a compound of formula (II), or a pharmaceutically acceptable salt thereof, comprising reacting a compound of formula (I), or a pharmaceutically acceptable salt thereof, in the presence of a P(R²)₃ reagent and an azodicarboxylate or azodicarboxamide, in the presence of an organic solvent:

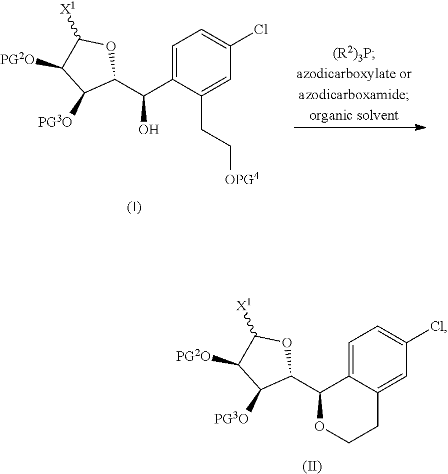

wherein X¹ is OH or OPG¹; PG¹ is a hydroxyl protecting group; PG², PG³ and PG⁴ are each independently H or a hydroxyl protecting group; or PG² and PG³ together with the oxygen atoms to which they are attached form a 1,2-dihydroxyl protecting group; and each R² is independently C₁-C₆alkyl or aryl.

In these embodiments, X¹ is OH or OPG¹. For example, in some aspects, X¹ is OH. In some aspects, X¹ is OPG¹ wherein PG¹ is a hydroxyl protecting group. As used herein, the term “hydroxyl protecting group” refers to a moiety that is bound to an oxygen atom of a compound (e.g., —O-PG²) such that the moiety (e.g., -PG²) can be removed under controlled conditions to yield a hydroxyl group (i.e., —OH). Hydroxyl protecting groups, methods of installing protecting groups, and methods for removing protecting groups are known to those of skill in the art and are described in, for example, Wuts, P. G. M., Greene's Protective Groups in Organic Synthesis, John Wiley & Sons, 5th ed. 2014. Preferred hydroxyl protecting groups include acid labile protecting groups that are known in the art. Acid labile protecting groups suitable for use in the methods of the disclosure include C₁₋₆alkyl. Also in these embodiments, PG², PG³ and PG⁴ are each independently H or a hydroxyl protecting group. In other embodiments, PG² and PG³ together with the oxygen atoms to which they are attached form a 1,2-dihydroxyl protecting group.

In some embodiments, PG¹, PG² PG³ and PG⁴ are each, independently, a hydroxyl protecting group that is stable to (i.e., not removed during reaction) nucleophiles.

In some embodiments, PG¹, PG² PG³ and PG⁴ are each, independently, a hydroxyl protecting group that is stable during reaction with other compounds. Exemplary nucleophile-stable hydroxyl protecting groups include alkyl ethers, benzyl ethers, substituted benzyl ethers (e.g., p-methoxybenzyl ether), and silyl ethers (e.g., t-butyldimethylsilyl ether, trimethylsilyl ether).

In some embodiments PG¹, PG² PG³ and PG⁴ are each, independently, an alkyl ether, such as, for example, a methyl ether, a methoxymethyl ether, a methylthiomethyl ether, a benzyloxymethyl ether, a substituted benzyloxymethyl ether, a t-butoxymethyl ether, a siloxymethyl ether, a methoxyethoxymethy ether, a tetrahydropyanyl ether, a 1-ethoxyethyl ether, a t-butyl ether, a trimethylsilyl ether, a t-butyldimethylsilyl ether, and the like.

In some embodiments PG¹, PG² PG³ and PG⁴ are each, independently, a methyl ether. In some embodiments PG² or PG³ is a methyl ether. In some embodiments, PG² is a methyl ether. In some embodiments, PG³ is a methyl ether.

In some embodiments, PG² and PG³ together with the oxygen atoms to which they are attached form a 1,2-dihydroxyl protecting group. In some embodiments, In some embodiments, PG² and PG³ are each, independently, a hydroxyl protecting group that is stable to nucleophiles. In some embodiments, PG² and PG³ together with the oxygen atoms to which they are attached form a 1,2-dihydroxyl protecting group that is stable during reaction with other compounds. Exemplary nucleophile-stable 1,2-dihydroxyl protecting groups include acetals (e.g., methylene acetal, ethylidene acetal, benzylidene acetal, p-methoxybenzylidene actetal, and the like), and ketals (e.g., acetonide and the like).

In some embodiments, PG² and PG³ together with the oxygen atoms to which they are attached form an acetonide protecting group.

In some aspects, PG² and PG³ is H or a hydroxyl protecting group; or PG² and PG³ together with the oxygen atoms to which they are attached form a 1,2-dihydroxyl protecting group.

In some embodiments, PG² is H. In other embodiments, PG² is a hydroxyl protecting group. In some embodiments, PG² is a nucleophile-stable hydroxyl protecting group. In some embodiments, PG² is a methoxymethyl ether, a methylthiomethyl ether, a benzyloxymethyl ether, a substituted benzyloxymethyl ether, a t-butoxymethyl ether, a siloxymethyl ether, a methoxyethoxymethy ether, a tetrahydropyanyl ether, a 1-ethoxyethyl ether, a t-butyl ether, a trimethylsilyl ether, a tbutyldimethylsilyl ether and the like.

In some embodiments, PG³ is H. In other embodiments, PG³ is a hydroxyl protecting group. In some embodiments, PG³ is a nucleophile-stable hydroxyl protecting group. In some embodiments, PG³ is a methoxymethyl ether, a methylthiomethyl ether, a benzyloxymethyl ether, a substituted benzyloxymethyl ether, a t-butoxymethyl ether, a siloxymethyl ether, a methoxyethoxymethy ether, a tetrahydropyanyl ether, a 1-ethoxyethyl ether, a t-butyl ether, a trimethylsilyl ether, a tbutyldimethylsilyl ether and the like.

In some embodiments, PG² and PG³ together with the oxygen atoms to which they are attached form a 1,2-dihydroxyl protecting group. 1,2-Dihydroxyl protecting groups are known in the art. See, e.g., Wuts, P. G. M., Greene's Protective Groups in Organic Synthesis, John Wiley & Sons, 5th ed. 2014. A suitable 1,2-dihydroxyl protecting group is acetonide.

In some embodiments, PG² and PG³ together with the oxygen atoms to which they are attached form a nucleophile-stable 1,2-dihydroxyl protecting group. In some embodiments, PG² and PG³ together with the oxygen atoms to which they are attached form a an acetonide protecting group.

Also, in these embodiments, each R² in the P(R²)₃ reagent is independently C₁-C₆alkyl or aryl. In some aspects, each R² in the P(R²)₃ reagent is independently C₁-C₆alkyl. In some aspects, each R² in the P(R²)₃ reagent is independently aryl. Examples of P(R²)₃ reagents suitable for use in the methods of the disclosure include trimethylphosphine, triethylphosphine, tri-n-propyl-phosphine, tri-n-butyl-phosphine, triphenylphosphine, (p-dimethylaminophenyl)diphenylphosphine, and diphenyl-2-pyridylphosphine. In some aspects, the P(R²)₃ reagent is trimethylphosphine. In some aspects, the P(R²)₃ reagent is triethylphosphine. In some aspects, the P(R²)₃ reagent is tri-n-propyl-phosphine. In some aspects, the P(R²)₃ reagent is tri-n-butyl-phosphine. In some aspects, the P(R²)₃ reagent is triphenylphosphine. In some aspects, the P(R²)₃ reagent is (p-dimethylaminophenyl) diphenylphosphine. In some aspects, the P(R²)₃ reagent is diphenyl-2-pyridylphosphine.

The methods of the disclosure use an azodicarboxylate or azodicarboxamide to produce compounds of formula (II). Azodicarboxylates include a R—C(O)—N═N—C(O)—O—R′ group and suitable azodicarboxylates are known in the art. Examples of azodicarboxylates or azodicarboxamides suitable for use in the described methods include diisopropylazodi-carboxylate (DIAD), tetramethyl azodicarboxamide (TMAD), and diethyl-azodicarboxylate (DEAD). In some aspects, the azodicarboxylate or azodicarboxamide is DIAD. In some aspects, the azodicarboxylate or azodicarboxamide is TMAD. In some aspects, the azodi-carboxylate or azodicarboxamide is DEAD. Mixtures of azodicarboxylates and azodi-carboxamides can also be used.

Organic solvents suitable for the production of compounds of formula (II) are known in the art. Suitable organic solvents include, for example, halogenated solvents such as dichloromethane, ethereal solvents such as diethyl ether, t-butyl methyl ether, tetrahydrofuran, and combinations thereof.

In some embodiments, the compound of formula (I) is a compound of formula (Ia) or a pharmaceutically acceptable salt thereof and the compound of formula (II) is a compound of formula (IIa) or a pharmaceutically acceptable salt thereof:

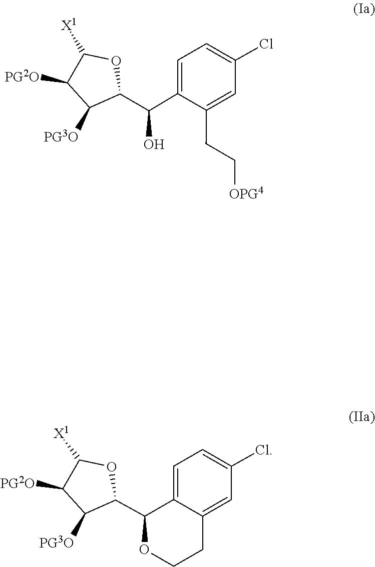

In some aspects, the disclosure is directed to processes for preparing a compound of formula (IIa), or a pharmaceutically acceptable salt thereof, comprising reacting a compound of formula (Ia), or a pharmaceutically acceptable salt thereof, in the presence of a P(R²)₃ reagent and an azodicarboxylate or azodicarboxamide, in the presence of an organic solvent:

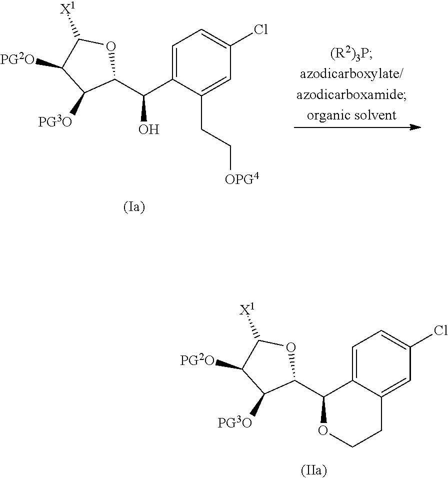

In some embodiments, the compound of formula (I) is a compound of formula (Ib) or a pharmaceutically acceptable salt thereof and the compound of formula (II) is a compound of formula (IIb) or a pharmaceutically acceptable salt thereof:

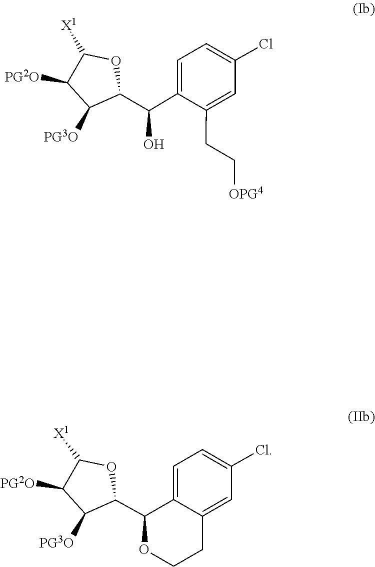

In some aspects, the disclosure is directed to processes for preparing a compound of formula (IIb), or a pharmaceutically acceptable salt thereof, comprising reacting a compound of formula (Ib), or a pharmaceutically acceptable salt thereof, in the presence of P(R²)₃ reagent and an azodicarboxylate in the presence of an organic solvent:

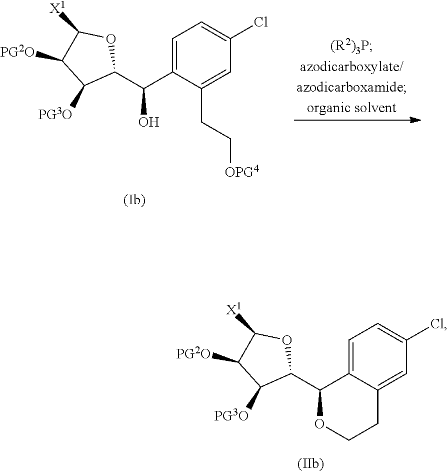

In some embodiments of the processes of the disclosure, the compound of formula (Ia), or a pharmaceutically acceptable salt thereof, is a compound of formula (Ia-1), or a pharmaceutically acceptable salt thereof:

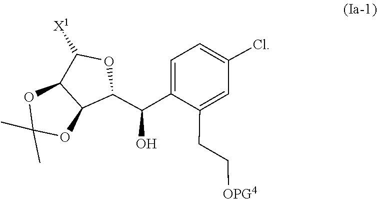

In some embodiments of the processes of the disclosure, the compound of formula (Ib), or a pharmaceutically acceptable salt thereof, is a compound of formula (lb-1):

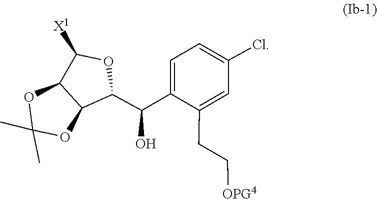

In the processes of the disclosure, the azodicarboxylate or azodicarboxamide used for reacting the compound of formula (Ia) (or formula (Ib)) to produce a compound of formula (IIa) (or formula (IIb)), is any azodicarboxylate or azodicarboxamide known in the art. Suitable azodicarboxylates or azodicarboxamides include those known to be generally useful in a Mitsunobu reaction. In some embodiments, the azodicarboxylate or azodi-carboxamide is DEAD, DIAD, or TMAD, or a mixture thereof. In some of these embodiments, the azodicarboxylate or azodicarboxamide is DIAD. In some of these embodiments, the azodicarboxylate or azodicarboxamide is TMAD. In some of these embodiments, the azodicarboxylate or azodicarboxamide is DEAD.

In the processes of the disclosure, the P(R²)₃ reagent used for reacting the compound of formula (Ia) or formula (Ib) to produce a compound of formula (IIa) or formula (IIb), respectively, is any phosphine known to be useful in synthetic organic chemistry. Suitable phosphines include those known to be generally useful in a Mitsunobu reaction. In some embodiments, the phosphine is (R²)₃P wherein R² is C₁-C₆alkyl, such as, for example, trimethylphosphine, triethylphosphine, tri-n-propylphosphine, tri-n-butyl-phosphine, and the like. Tri-n-butylphosphine, (n-Bu)₃P, is one exemplary phosphine reagent. In other embodiments, the phosphine is (R²)₃P wherein R² is aryl, such as, for example, triphenylphosphine, (p-dimethylaminophenyl)diphenylphosphine, diphenyl-2-pyridylphosphine, and the like. Triphenylphosphine is one exemplary phosphine reagent.

In the processes of the disclosure, the organic solvent used for reacting the compound of formula (Ia) or formula (Ib) to produce a compound of formula (IIa) or formula (IIb), respectively, is any organic solvent known to be generally suitable for use in a Mitsunobu reaction. In some embodiments, the organic solvent is dichloromethane, chloroform, tetrahydrofuran, dioxane, diisopropylether, DMF, acetonitrile, or a mixtures thereof. In some embodiments, the organic solvent is dichloromethane. In some embodiments, the organic solvent is tetrahydrofuran. In other embodiments, the organic solvent is a mixture of dichloromethane and tetrahydrofuran.

In some embodiments, the organic solvent used for reacting the compound of formula (Ia) or formula (Ib) to produce a compound of formula (IIa) or formula (IIb), respectively, is an aprotic organic solvent. Exemplary aprotic organic solvents include Perfluorohexane, α,α,α-trifluorotoluene, pentane (Pent), hexane (Hex), cyclohexane (Cy), methylcyclohexane, decalin [c+t], dioxane, carbon tetrachloride, freon-11, benzene, toluene, triethyl amine, carbon disulfide, diisopropyl ether, diethyl ether (ether), t-butyl methyl ether (MTBE), chloroform, ethyl acetate, 1,2-dimethoxy-ethane (glyme), 2-methoxyethyl ether (diglyme), tetrahydrofuran (THF), methylene chloride, pyridine (Py), 2-butanone (MEK), acetone, hexamethylphosphoramide (HMPA), N-methyl-pyrrolidinone (NMP), nitromethane, dimethylformamide (DMF), acetonitrile, sulfolane, dimethyl sulfoxide (DMSO), propylene carbonate, and mixtures thereof.

In some embodiments, the aprotic organic solvent is diethyl ether, t-butyl methyl ether, or tetrahydrofuran, or mixtures thereof.

In some embodiments, the aprotic organic solvent is diethyl ether.

In other embodiments, the aprotic organic solvent is t-butyl methyl ether.

In other embodiments, the aprotic organic solvent is tetrahydrofuran.

In some embodiments of the processes of the disclosure, the preparation of compounds of formula (II), for example, compounds of formula (IIa) or (IIb), are carried out in the presence of an additive. As used herein, the term “additive” refers to a compound or mixture of compounds that increases the yield, rate, or selectivity of the reaction. Additives suitable for use in the methods of the disclosure are those known to be generally useful in a Mitsunobu reaction.

In some embodiments, the temperature of the reaction mixture for reacting the compound of formula (Ia) or formula (Ib) to produce a compound of formula (IIa) or formula (IIb), respectively, is between about 0° C. and about 50° C. In some embodiments, the temperature is ambient temperature. In some embodiments, the temperature is about 25° C. In other embodiments, the temperature is between about 10° C. to about 25° C. or between about 20° C. to about 25° C. In some aspects, the temperature is 15, 16, 17, 28, 19, 20, 21, 2, 23, 24, 25, 26, 27, 28, 29, or 30° C.

In some embodiments, the compound of formula (Ia), or a pharmaceutically acceptable salt thereof, is a compound of formula (Ia-1) or a pharmaceutically acceptable salt thereof.

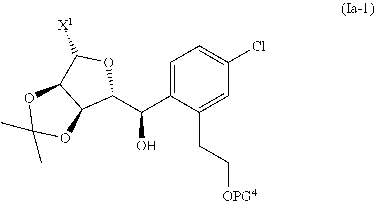

In some embodiments, the compound of formula (Ib), or a pharmaceutically acceptable salt thereof, is a compound of formula (Ib-1), or a pharmaceutically acceptable salt thereof.

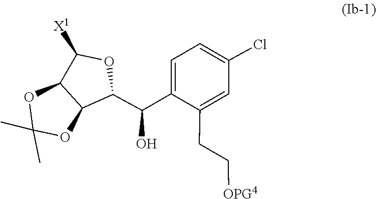

In some embodiments, the compound of formula (IIa), or a pharmaceutically acceptable salt thereof, is a compound of formula (IIa-1) or a pharmaceutically acceptable salt thereof.

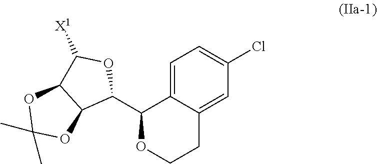

In some embodiments, the compound of formula (IIb), or a pharmaceutically acceptable salt thereof, is a compound of formula (IIb-1), or a pharmaceutically acceptable salt thereof.

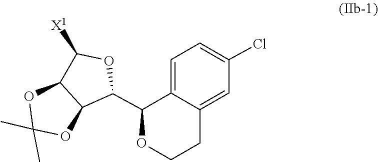

It will be clear to those of skill in the art that reacting the compound of formula (I) (e.g., formula (Ia), formula (Ia-1) formula (Ib), formula (Ib-1), or a mixture thereof), or a pharmaceutically acceptable salt thereof, to produce the compound of formula (II) (e.g., formula (IIa), formula (IIa-1), formula (IIb) formula (IIba-1)), or a pharmaceutically acceptable salt thereof, results in the formation of an asymmetric cyclopentyl carbon atom (*).

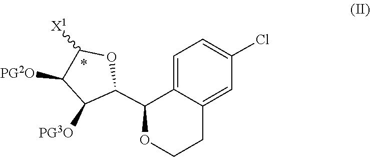

In some embodiments, production of the compound of formula (II), or a pharmaceutically acceptable salt thereof, results in an enantiomeric excess at the cyclopentyl carbon atom (*) of at least 80%; at least 90%; at least 95%; at least 98%; at least 99%; at least 99.5%; at least 99.8%; or at least 99.9%. As used herein, the term “enantiomeric excess” refers to the difference between the amount of one enantiomer at the (*)carbon minus the amount of the other enantiomer at the (*) carbon. A racemic mixture has an enantiomeric excess of 0%. A single enantiomer has an enantiomeric excess of 100%. For example, if a reaction produces 99% of enantiomer 1 and 1% of enantiomer 2, then the enantiomeric excess is 98%. Methods for determining the enantiomeric excess at the (*) carbon atom will be known to those of skill in the art and include, for example, HPLC using a chiral stationary phase.

Compounds of the disclosure that include more than one chiral stereocenter can be produced in diastereomeric excess, that is, enriched in one diastereomer over the other possible diastereomers. In some embodiments, compounds of formula (II) produced according to the methods disclosed herein are produced in a diastereomeric excess, that is enriched in a particular diastereomer. In some embodiments, the diastereomeric excess is at least 80%; at least 90%; at least 95%; at least 98%; at least 99%; at least 99.5%; at least 99.8%; or at least 99.9%. Methods for determining the diastereomeric excess will be known to those of skill in the art and include, for example, HPLC using a chiral or achiral stationary phase.

In some embodiments of the disclosed processes, X¹ of formula (I) is OPG¹, as shown by the compound of formula (III), or a pharmaceutically acceptable salt thereof:

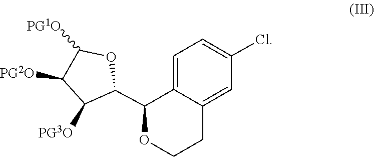

In some embodiments, the compound of formula (III) is a compound of formula (IIIa) or a compound of formula (IIIb), or a pharmaceutically acceptable salt thereof:

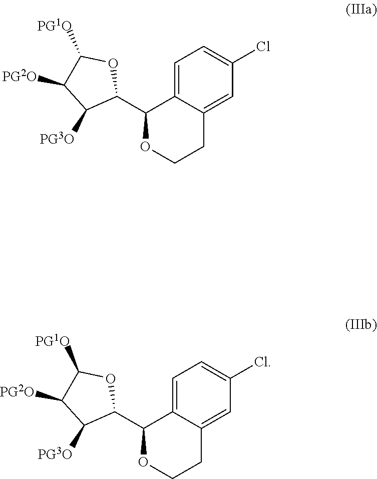

In some embodiments of the processes of the disclosure, the compound of formula (IIIa) or formula (IIIb), or a pharmaceutically acceptable salt thereof, is a compound of formula (IIIa-1) or formula (IIIb-1), or a pharmaceutically acceptable salt thereof:

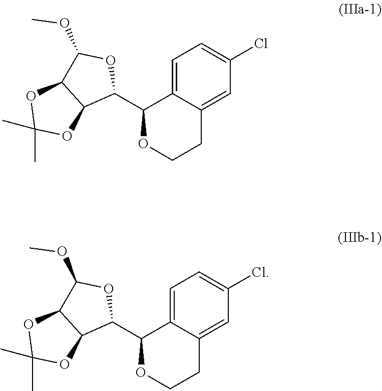 [SMILES: CO[C@@H]1O[C@H]([C@@H]2OCCc3cc(Cl)ccc32)[C@H]2OC(C)(C)O[C@@H]12 ; CO[C@H]1O[C@H]([C@@H]2OCCc3cc(Cl)ccc32)[C@H]2OC(C)(C)O[C@@H]12]

In some embodiments of the processes of the disclosure, the compound of formula (IV), or a pharmaceutically acceptable salt thereof, is produced by reacting a compound of formula (III), or a pharmaceutically acceptable salt thereof, with an aqueous acid capable of hydrolyzing hydroxyl protecting groups. Such acids will depend on the identities of the hydroxyl protecting groups (PG), and, will be known to those skilled in the art as described in, for example, Wuts, P. G. M., Greene's Protective Groups in Organic Synthesis, John Wiley & Sons, 5th ed. 2014.

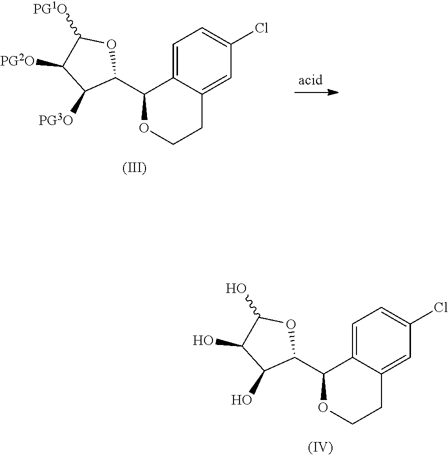

Aqueous acids of the disclosure include a mixture of water and an acid capable of hydrolyzing hydroxyl protecting groups. Examples of suitable acids include mineral acids, for example, HCl, H₃PO₄, H₂SO₄, and mixtures thereof. In some aspects, the acid is HCl. In some aspects, the acid is H₃PO₄. In some aspects, the acid is H₂SO₄. In other embodiments, the acid is an acidic ion-exchange resin. Non-limiting examples of such resins include those sold under the tradenames Dowex (styrene divinylbenzene (gel) with sulfonic acid functional groups); Amberlite (Styrene-Divinylbenzene (DVB) gel or macroreticular, with sulfonic acid functional groups); and Amberlyst (styrene-divinylbenzene (macroreticular) with sulfonic acid functional groups). In some embodiments, the aqueous acid is used in the presence of an organic solvent. Non-limiting examples of organic solvents that may be used in this regard include acetonitrile (ACN), THF, DMF, and alcohols such as methanol, ethanol and isopropanol. In some embodiments, the aqueous acid is a mixture of H₂SO₄, water, and ACN. In other embodiments, the aqueous acid is a mixture of water, acidic ion-exchange resin, and optionally an organic solvent.

In some embodiments, the compound of formula (IV) is a compound of formula (IVa) or a compound of formula (IVb), or a pharmaceutically acceptable salt thereof:

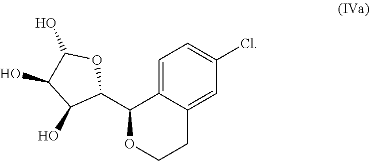

In some embodiments of the processes of the disclosure, the compound of formula (IVa), or a pharmaceutically acceptable salt thereof, is produced by reacting a compound of formula (IIIa), or a pharmaceutically acceptable salt thereof, with an aqueous acid:

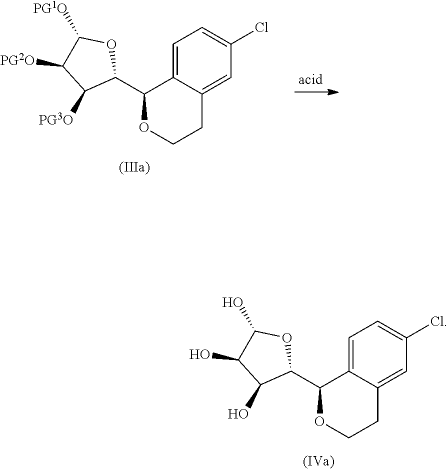

In some embodiments of the processes of the disclosure, the compound of formula (IVa), or a pharmaceutically acceptable salt thereof, is produced by reacting a compound of formula (IIIa-1), or a pharmaceutically acceptable salt thereof, with an aqueous acid:

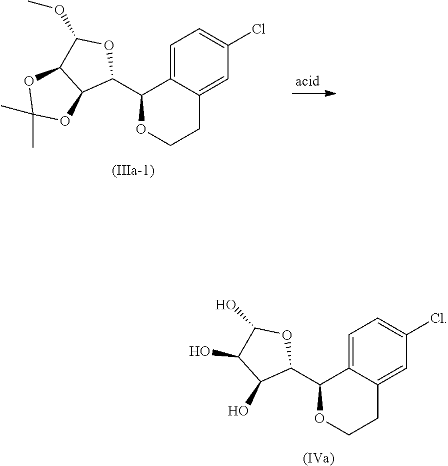

In some embodiments of the processes of the disclosure, the compound of formula (IV), or a pharmaceutically acceptable salt thereof, is produced by reacting a compound of formula (IIIa), or a pharmaceutically acceptable salt thereof, with an aqueous acid:

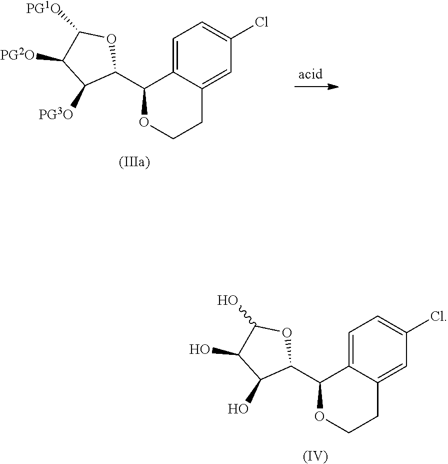

In some embodiments of the processes of the disclosure, the compound of formula (IV), or a pharmaceutically acceptable salt thereof, is produced by reacting a compound of formula (IIIa-1), or a pharmaceutically acceptable salt thereof, with an aqueous acid:

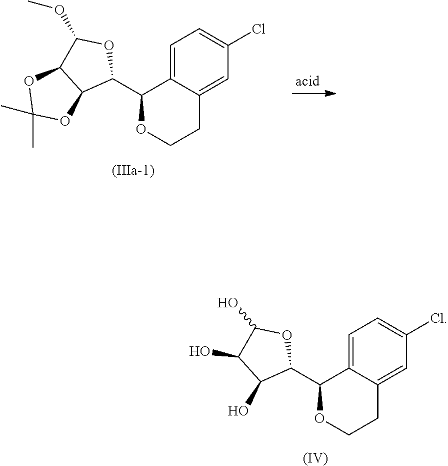

In some embodiments of the processes of the disclosure, the compound of formula (IVb), or a pharmaceutically acceptable salt thereof, is produced by reacting a compound of formula (IIIb), or a pharmaceutically acceptable salt thereof, with an aqueous acid:

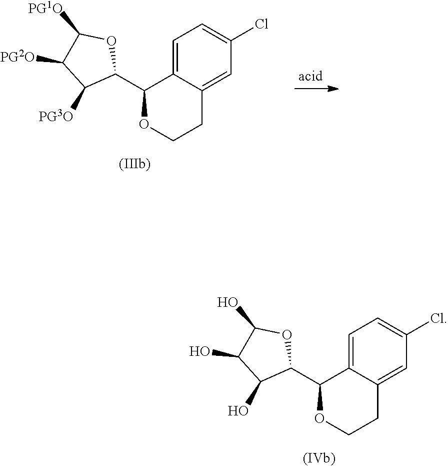

In some embodiments of the processes of the disclosure, the compound of formula (IVb), or a pharmaceutically acceptable salt thereof, is produced by reacting a compound of formula (IIIb-1), or a pharmaceutically acceptable salt thereof, with an aqueous acid:

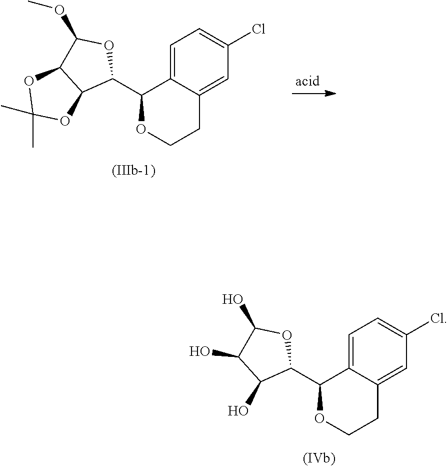

In some embodiments, the reaction to produce a compound of formula (IV), or a pharmaceutically acceptable salt thereof, is conducted at a temperature between about 0° C. and about 20° C., for example, 0, 1, 2, 3, 4, 5, 6, 7, 8, 9, 10, 11, 12, 13, 14, 15, 16, 17, 18, 19, or 20° C. In some aspects, the reaction is conducted at a temperature that is between 0-5° C.

In some embodiments of the processes of the disclosure, the disclosure is directed to processes for preparing a compound of formula (VI), or a pharmaceutically acceptable salt thereof, comprising reacting a compound of formula (IV), or a pharmaceutically acceptable salt thereof, with a compound of formula (V), or a pharmaceutically acceptable salt thereof, in the presence of a P(R²)₃ reagent and an azodicarboxylate or azodicarboxamide, in the presence of an organic solvent:

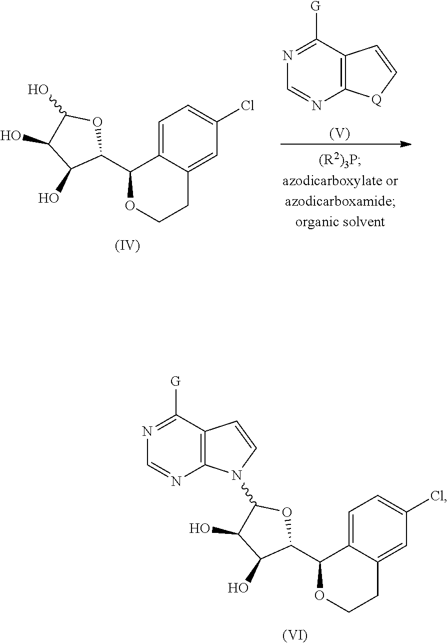

wherein G is a halogen or C₁-C₆alkyl; each R² is independently C₁-C₆alkyl or aryl; and Q is —N—H or N⁻K⁺.

In some aspects, G is a halogen, for example, F, Cl, Br, or I. A preferred halogen is Cl. In some aspects, G is C₁-C₆alkyl, for example, methyl, ethyl, propyl, i-propyl, and the like. A preferred C₁-C₆alkyl is methyl.

In some aspects, Q is N—H. In some aspects, Q is N−K⁺. In some aspects, Q is N—Li⁺. In some aspects, Q is N—Na⁺. In some aspects, Q is N−Cs⁺.

In some embodiments, the compound of formula (VI) is a compound of formula (VIa) or a compound of formula (VIb), or a pharmaceutically acceptable salt thereof:

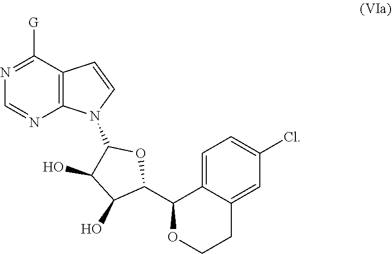

In some embodiments of the processes of the disclosure, the disclosure is directed to processes for preparing a compound of formula (VIa), or a pharmaceutically acceptable salt thereof, comprising reacting a compound of formula (IV), or a pharmaceutically acceptable salt thereof, with a compound of formula (V), or a pharmaceutically acceptable salt thereof, in the presence of a P(R²)₃ reagent, an azodicarboxylate or azodicarboxamide, in the presence of an organic solvent:

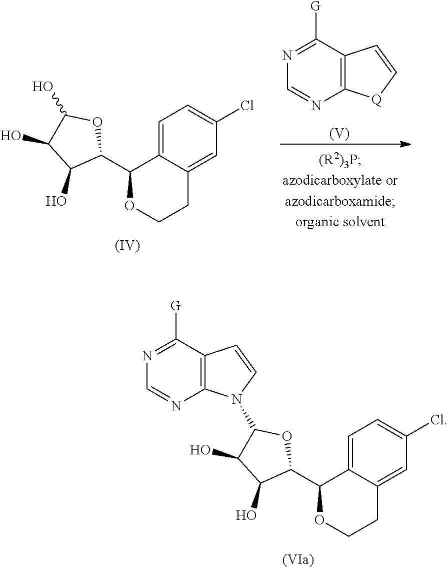

In some embodiments of the processes of the disclosure, the disclosure is directed to processes for preparing a compound of formula (VIa), or a pharmaceutically acceptable salt thereof, comprising reacting a compound of formula (IVa), or a pharmaceutically acceptable salt thereof, with a compound of formula (V), or a pharmaceutically acceptable salt thereof, in the presence of a P(R²)₃ reagent and an azodicarboxylate or azodicarboxamide in the presence of an organic solvent:

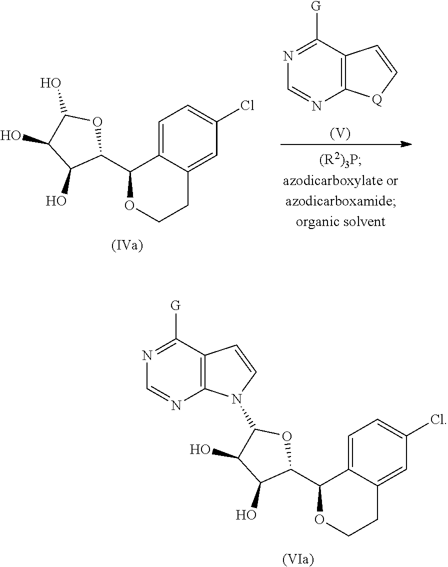

In some embodiments of the processes of the disclosure, the disclosure is directed to processes for preparing a compound of formula (VIb), or a pharmaceutically acceptable salt thereof, comprising reacting a compound of formula (IVb), or a pharmaceutically acceptable salt thereof, with a compound of formula (V), or a pharmaceutically acceptable salt thereof, in the presence of a P(R²)₃ reagent and, an azodicarboxylate or azodicarboxamide, in the presence of an organic solvent:

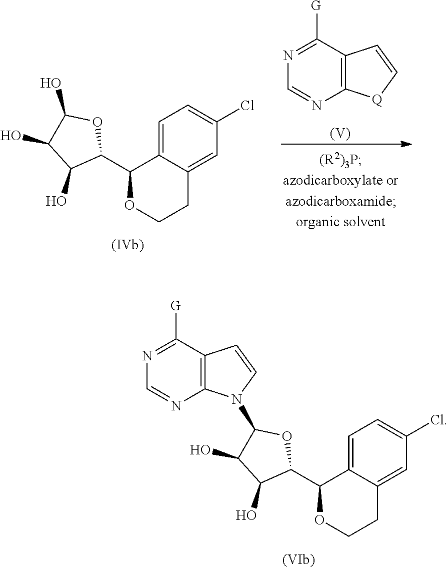

In some embodiments, the reaction of a compound of formula (IV) with a compound of formula (V) proceeds through an epoxide intermediate having the formula IVaa:

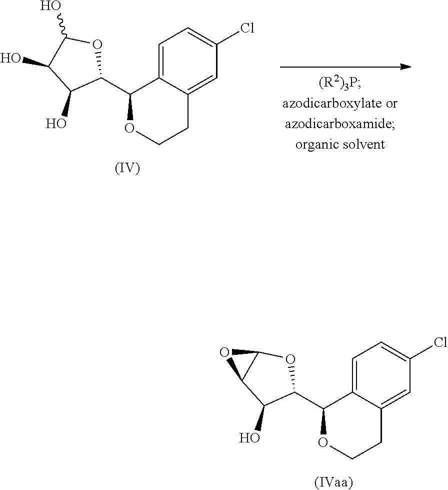

In some embodiments, the compound of formula (IVaa) is isolated prior to reaction with a compound of formula (V).

In some aspects, the processes of the disclosure for preparing the compound of formula (VIa) further comprise converting the compound of formula (IV) to an epoxide of formula (IVaa) by reacting the compound of formula (IV) with a phosphine, an azodicarboxylate or azodicarboxamide, in the presence of an appropriate organic solvent:

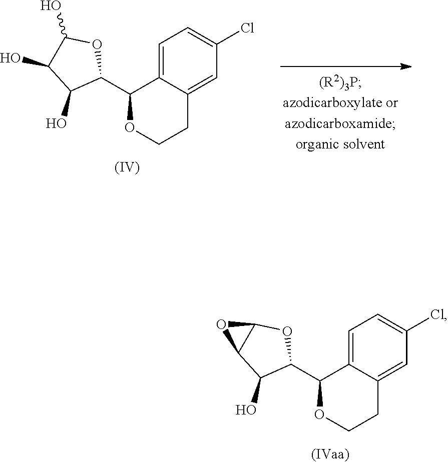

wherein each R² is independently C₁-C₆alkyl or aryl.

In some embodiments, the organic solvent is an aprotic organic solvent.

In some embodiments, the processes further comprise reacting the compound of formula (IVaa) with a compound of formula (V), or a basic salt thereof, in an organic solvent to give a compound of formula (VIa):

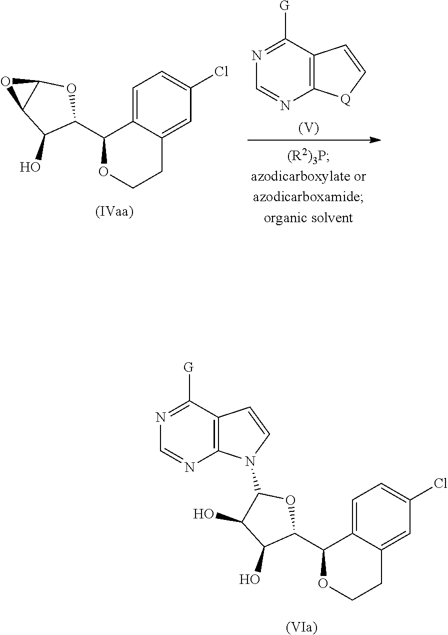

wherein G is a halogen or C₁-C₆alkyl; each R² is independently C₁-C₆alkyl or aryl; and Q is —N—H or N⁻K⁺.

In embodiments wherein G is —Cl, the compound of formula (V), or a pharmaceutically acceptable salt thereof, is the compound of formula (Va), or a pharmaceutically acceptable salt thereof:

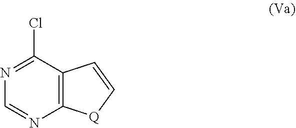

wherein Q is —N—H or N⁻K⁺. In some embodiments of compounds of formula (Va), Q is N—H. In some embodiments of compounds of formula (Va), Q is N—K⁺.

In embodiments wherein Q is N—K⁺, compound (V) is prepared from Compound (V) wherein Q is N—H by mixing with a suitable base in an organic solvent. The suitable base may be any base capable of creating the N—K⁺ salt, such as potassium tert-butoxide or potassium hydroxide.

In embodiments wherein G is methyl, the compound of formula (V), or a pharmaceutically acceptable salt thereof, is the compound of formula (Vb), or a pharmaceutically acceptable salt thereof:

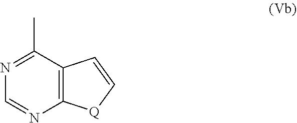

wherein Q is —N—H or N⁻K⁺. In some embodiments of compounds of formula (Vb), Q is N—H. In some embodiments of compounds of formula (Vb), Q is N—K⁺.

In embodiments wherein G is —Cl, the compound of formula (VIa), or a pharmaceutically acceptable salt thereof, is the compound of formula (VIa-2), or a pharmaceutically acceptable salt thereof:

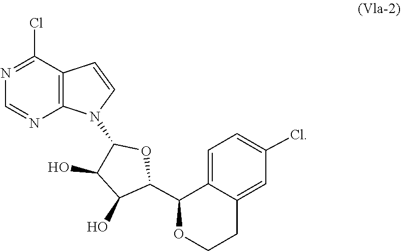 [SMILES: O[C@@H]1[C@H](O)[C@@H]([C@@H]2OCCc3cc(Cl)ccc32)O[C@H]1n1ccc2c(Cl)ncnc21]

In embodiments wherein G is methyl, the compound of formula (VIa), or a pharmaceutically acceptable salt thereof, is the compound of formula (VIa-1), or a pharmaceutically acceptable salt thereof:

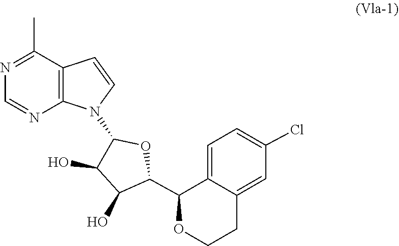 [SMILES: Cc1ncnc2c1ccn2[C@@H]1O[C@H]([C@@H]2OCCc3cc(Cl)ccc32)[C@@H](O)[C@H]1O]

In embodiments wherein G is methyl, the compound of formula (VIa), or a pharmaceutically acceptable salt thereof, is the compound of formula (VIa-3), or a pharmaceutically acceptable salt thereof:

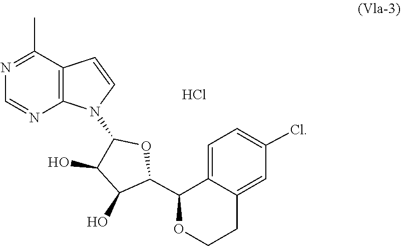 [SMILES: Cc1ncnc2c1ccn2[C@@H]1O[C@H]([C@@H]2OCCc3cc(Cl)ccc32)[C@@H](O)[C@H]1O ; Cl]

In embodiments of the disclosed processes in which the compound of formula (VIa-1) is isolated as a solid salt (such as, for example, a HCl salt, a phosphate salt, a sulfate salt, oxalate salt, oxalate salt, maleate salt), the salt may be converted to the formula (VIa-1) free base by reaction with a suitable base in the presence of an appropriate solvent. In some embodiments, the free base of formula (VIa-1) is obtained by treating the HCl salt of formula (VIa-3) with aqueous base, such as, for example, aqueous ammonium hydroxide:

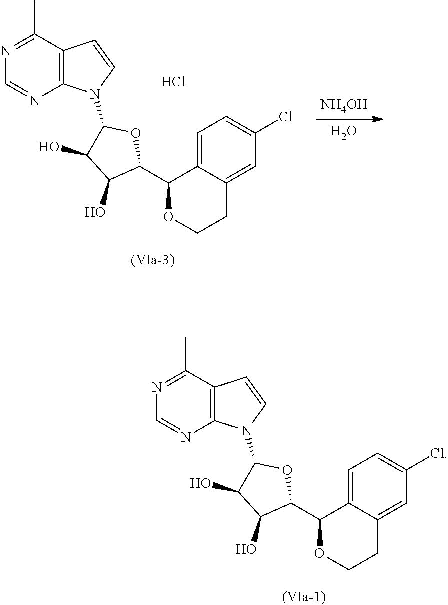

In some embodiments, this conversion is conducted at a temperature from about 10° C. to about 50° C., for example, about 5, 10, 15, 20, 25, 30, 35, 40, 45, or 50° C., preferably from about 15° C. to about 35° C.

In some embodiments, the processes of the disclosure comprise reacting the compound of formula (VIa-2), or a pharmaceutically acceptable salt thereof, with a Grignard reagent in the presence of a catalyst to give the compound of formula (VIa-1), or a pharmaceutically acceptable salt thereof:

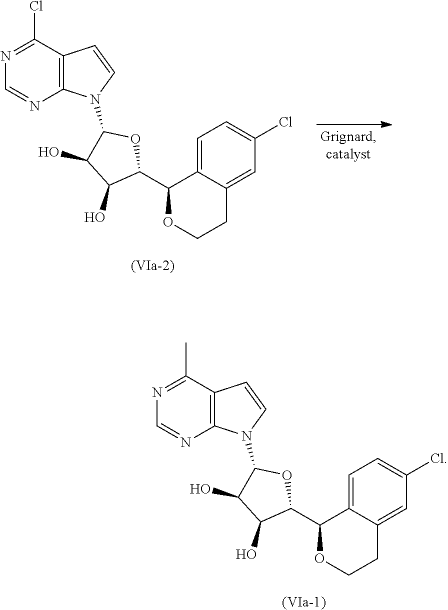 [SMILES: O[C@@H]1[C@H](O)[C@@H]([C@@H]2OCCc3cc(Cl)ccc32)O[C@H]1n1ccc2c(Cl)ncnc21 ; "Grignard,catalyst" ; "(VIa-2)" ; Cc1ncnc2c1ccn2[C@@H]1O[C@H]([C@@H]2OCCc3cc(Cl)ccc32)[C@@H](O)[C@H]1O ; "(VIa-1)"]

Suitable catalysts are known in the art. In an embodiments, a suitable catalyst is M(acac)₃ wherein M is Fe. Suitable Grignard reagents are known in the art and can include any suitable methyl-nucleophile combining with suitable catalyst. In some embodiments, the Grignard reagent is MeMgBr.

In embodiments wherein G is —Cl, the compound of formula (VIb), or a pharmaceutically acceptable salt thereof, is the compound of formula (VIb-2), or a pharmaceutically acceptable salt thereof:

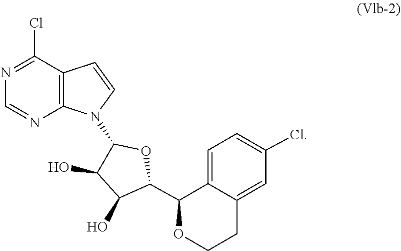 [SMILES: O[C@@H]1[C@H](O)[C@@H]([C@@H]2OCCc3cc(Cl)ccc32)O[C@H]1n1ccc2c(Cl)ncnc21]

In embodiments wherein G is methyl, the compound of formula (VIb), or a pharmaceutically acceptable salt thereof, is the compound of formula (VIb-1), or a pharmaceutically acceptable salt thereof:

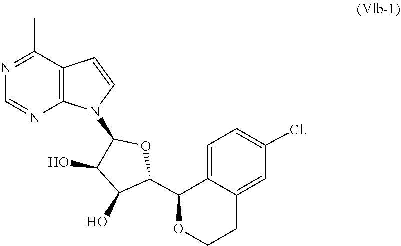 [SMILES: Cc1ncnc2c1ccn2[C@H]1O[C@H]([C@@H]2OCCc3cc(Cl)ccc32)[C@@H](O)[C@H]1O]

In embodiments wherein G is methyl, the compound of formula (VIb), or a pharmaceutically acceptable salt thereof, is the compound of formula (VIb-3), or a pharmaceutically acceptable salt thereof:

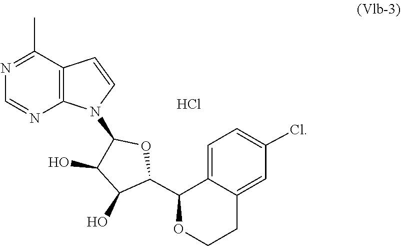 [SMILES: Cc1ncnc2c1ccn2[C@H]1O[C@H]([C@@H]2OCCc3cc(Cl)ccc32)[C@@H](O)[C@H]1O ; Cl]

In embodiments of the disclosed processes in which the compound of formula (VIb-1) is isolated as a solid salt (such as, for example, a HCl salt, a phosphate salt, a sulfate salt, oxalate salt, oxalate salt, maleate salt), the salt may be converted to the formula (VIb-1) free base by reaction with a suitable base in the presence of an appropriate solvent. In some embodiments, the free base of formula (VIb-1) is obtained by treating the HCl salt of formula (VIb-3) with aqueous base, such as, for example, aqueous ammonium hydroxide:

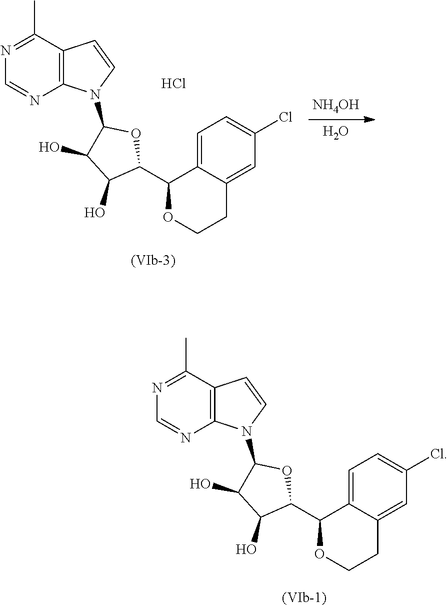

In some embodiments, this conversion is conducted at a temperature from about 10° C. to about 50° C., for example, about 5, 10, 15, 20, 25, 30, 35, 40, 45, or 50° C., preferably from about 15° C. to about 35° C.

In some embodiments, the processes of the disclosure comprise reacting the compound of formula (VIb-2), or a pharmaceutically acceptable salt thereof, with a Grignard reagent in the presence of a metal-acetylacetonate to give the compound of formula (VIb-1), or a pharmaceutically acceptable salt thereof:

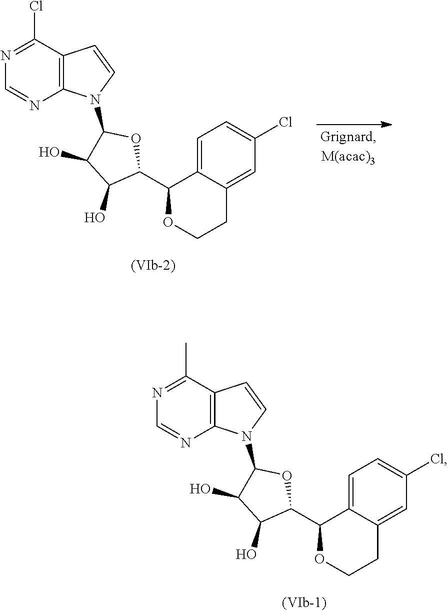 [SMILES: O[C@@H]1[C@H](O)[C@@H]([C@@H]2OCCc3cc(Cl)ccc32)O[C@@H]1n1ccc2c(Cl)ncnc21 ; "Grignard,M(acac)3" ; "(VIb-2)" ; Cc1ncnc2c1ccn2[C@H]1O[C@H]([C@@H]2OCCc3cc(Cl)ccc32)[C@@H](O)[C@H]1O ; "(VIb-1)"]

In some embodiments, M is Fe. Suitable Grignard reagents are known in the art. In some embodiments, the Grignard reagent is MeMgBr.

In some aspects, the processes of the disclosure further comprise contacting the compound of formula (VIa-1) with an acid to form a pharmaceutically acceptable salt of the compound of formula (VIa-1). In some embodiments, the pharmaceutically acceptable salt of the compound of formula (VIa-1) is a HCl salt, a phosphate salt, a sulfate salt, oxalate salt, oxalate salt, or maleate salt. The pharmaceutically acceptable salt may be prepared treating the compound of formula (VIa-1) with a suitable acid in the presence of suitable solvent.

In some embodiments, the processes of the disclosure further comprises reacting the compound of formula (VIa-1) with hydrochloric acid in a solvent to produce a pharmaceutically acceptable salt of the compound of formula (VIa-1) that is a compound of formula (VIa-3):

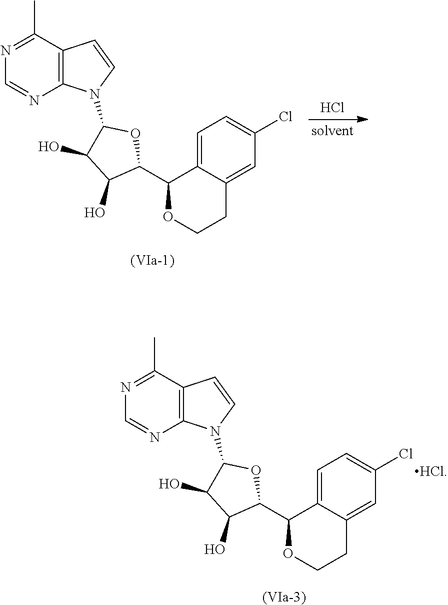

In some aspects, the processes of the disclosure further comprise reacting the compound of formula (VIb-1) with an acid to form a pharmaceutically acceptable salt of the compound of formula (VIb-1). In some embodiments, the pharmaceutically acceptable salt of the compound of formula (VIb-1) is a HCl salt, a phosphate salt, a sulfate salt, oxalate salt, oxalate salt, or maleate salt. The pharmaceutically acceptable salt may be prepared treating the compound of formula (VIb-1) with a suitable acid in the presence of suitable solvent.

In some embodiments, the processes of the disclosure further comprises contacting the compound of formula (VIb-1) with hydrochloric acid in a solvent to produce the pharmaceutically acceptable salt of the compound of formula (VIb-1) that is a compound of formula (VIb-3):

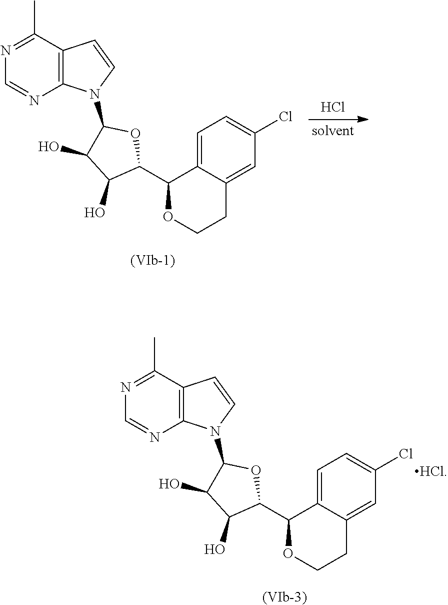

In some embodiments, the solvent used for the conversion of a compound of (VIa-1) or (VIb-1) to a compound of (VIa-3) or (VIb-3), respectively, is an alcohol, such as, for example methanol, ethanol, isopropanol, and the like, and mixtures thereof. In some embodiments, the solvent is ethanol. In some embodiments, the conversion is conducted at a temperature of from about 0° C. to about 75° C., for example, 0, 5, 10, 15, 20, 25, 30, 35, 40, 45, 50, 55, 60, 65, 70, or 75° C., preferably between about 35-50° C.

In some embodiments, the disclosure is directed to a process for preparing a compound of formula (VIa-1), formula (VIa-2), formula (VIa-3), formula (VIb-1), formula (VIb-2) or (VIb-3), or a pharmaceutically acceptable salt thereof, wherein the process comprises any process disclosed herein.

In some aspects, the processes of the disclosure provide the compound of formula (VIa-1), or a pharmaceutically acceptable salt thereof, such as a compound of (VIa-3), or a compound of (VIa-2), in high stereoisomeric purity. That is, the compound of formula (VIa-1), or a pharmaceutically acceptable salt thereof, such as a compound of (VIa-3), or a compound of (VIa-2), is obtained predominantly as the stereoisomer having the absolute configuration shown below:

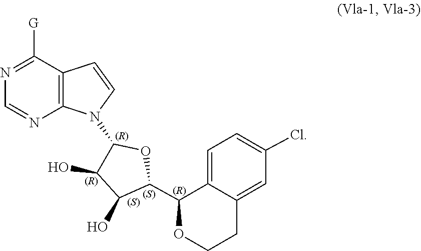

In some embodiments, the diastereomeric excess in the compound of formula (VIa-1) or a pharmaceutically acceptable salt thereof, such as a compound of (VIa-3), or a compound of (VIa-2), is at least 80%; at least 90%; at least 95%; at least 98%; at least 99%; at least 99.5%; at least 99.8%; or at least 99.9%. In some embodiments, the enantiomeric excess in the compound of formula (VIa-1) or a pharmaceutically acceptable salt thereof, such as a compound of (VIa-3), or a compound of (VIa-2), is at least 80%; at least 90%; at least 95%; at least 98%; at least 99%; at least 99.5%; at least 99.8%; or at least 99.9%. Methods of determining diastereomeric excess and enantiomeric excess are known to those skilled in the art, and include, for example, HPLC methods such as those known in the art and described herein.

In some aspects, the processes of the disclosure provide the compound of formula (VIb), or a pharmaceutically acceptable salt thereof, such as a compound of (VIb-1) or a compound of (VIb-3), or a compound of (VIb-2), in high stereoisomeric purity. That is, the compound of formula (VIb-1), or a pharmaceutically acceptable salt thereof, such as a compound of (VIb-3), or a compound of (VIb-2), or a pharmaceutically acceptable salt thereof, is obtained predominantly as the stereoisomer having the absolute configuration shown below:

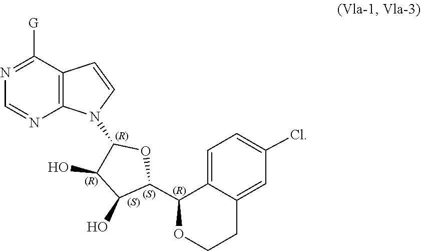

In some embodiments, the diastereomeric excess in the compound of formula (VIb-1) or a pharmaceutically acceptable salt thereof, such as a compound of (VIb-3) or a compound of (VIb-2), is at least 80%; at least 90%; at least 95%; at least 98%; at least 99%; at least 99.5%; at least 99.8%; or at least 99.9%. In some embodiments, the enantiomeric excess in the compound of formula (VIb-1) or a pharmaceutically acceptable salt thereof, such as a compound of (VIb-3), or a compound of (VIb-2), is at least 80%; at least 90%; at least 95%; at least 98%; at least 99%; at least 99.5%; at least 99.8%; or at least 99.9%. Methods of determining diastereomeric excess and enantiomeric excess are known to those skilled in the art, and include, for example, HPLC methods such as those described in the art and herein.

In some embodiments, the processes of the disclosure comprises the following steps to produce a compound of formula (VIa), such as formula (VIa-1), or formula (VIa-2), or formula (VIa-3).

A compound of formula (XXI), or a pharmaceutically acceptable salt thereof, is reacted with suitable Grignard reagent to produce a compound of formula (XXI′), or a pharmaceutically acceptable salt thereof:

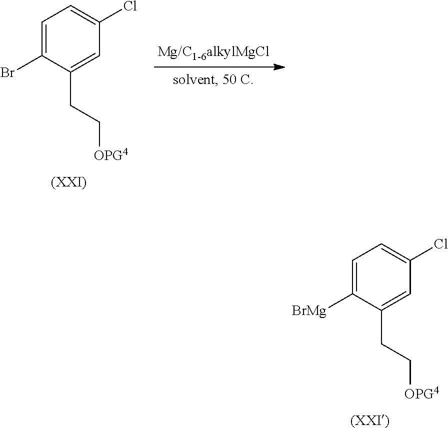

wherein PG⁴ is as defined with respect to formula (I). In a compound of formula (XXI), Br may be replaced by I.

In certain embodiments, PG⁴ of the compound of formula (XXI) and formula (XXI′) is a hydroxyl protecting group. In certain embodiments, PG⁴ of the compound of formula (XXI) and formula (XXI′) is tetrahydropyran (THP). In certain embodiments, the C₁₋₆alkyl of “C₁₋₆alkylMgCl” is methyl or ethyl.

In certain embodiments, the compound of formula (XXI) is compound 10 shown below:

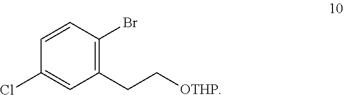

In certain embodiments, the compound of formula (XXI′) is compound 10′ shown below:

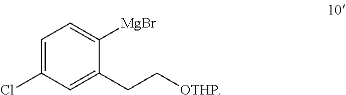

A compound of formula (XXI′), or a pharmaceutically acceptable salt thereof, is reacted with a compound of formula (XXII) (or the corresponding aldehyde thereof or an adduct thereof), or a pharmaceutically acceptable salt thereof, to produce a compound of formula (XXIII), or a pharmaceutically acceptable salt thereof:

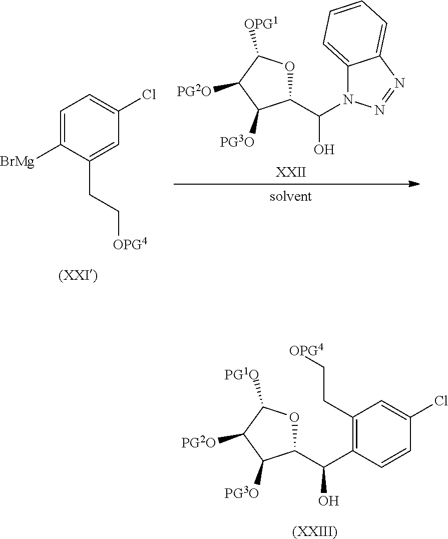

wherein PG¹, PG², PG³ and PG⁴ are as defined with respect to formula (I).

In certain embodiments, PG¹, PG², PG³ and PG⁴ of the compound of formula (XXI) and formula (XXI′) are each, independently, a hydroxyl protecting group; or PG² and PG³ together with the oxygen atoms to which they are attached form a 1,2-dihydroxyl protecting group. In certain embodiments, PG⁴ of the compound of formula (XXI) and formula (XXIII) is tetrahydropyran (THP).

In certain embodiments, a compound of formula (XXIII) is represented by a compound of formula (Ia) or a compound of formula (Ia-1).

In certain embodiments, the compound of formula (XXII) is compound 20 shown below:

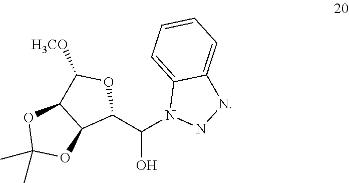

In certain embodiments, the compound of formula (XXIII) is compound 30 shown below:

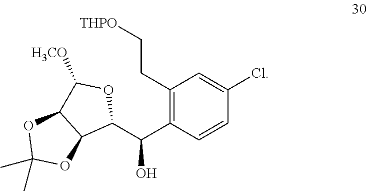

A compound of formula (XXIII), or a pharmaceutically acceptable salt thereof, is reacted with an alcohol to produce a compound of formula (XXIII′), or a pharmaceutically acceptable salt thereof:

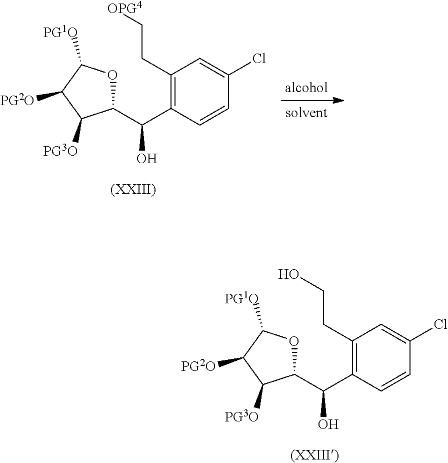

wherein PG¹, PG², PG³ and PG⁴ are as defined with respect to formula (I).

In certain embodiments, PG¹, PG², PG³ and PG⁴ of the compound of formula (XXIII) and PG¹, PG², and PG³ of the compound of formula (XXIII′) are each, independently, a hydroxyl protecting group; or PG² and PG³ together with the oxygen atoms to which they are attached form a 1,2-dihydroxyl protecting group. In certain embodiments, PG⁴ of the compound of formula (XXIII) is tetrahydropyran (THP).

In certain embodiments, a compound of formula (XXIII′) is represented by a compound of formula (Ia) or a compound of formula (Ia-1).

In certain embodiments, the compound of formula (XXIII′) is compound 40 shown below:

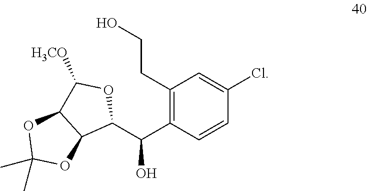 [SMILES: CO[C@@H]1O[C@H]([C@H](O)c2ccc(Cl)cc2CCO)[C@H]2OC(C)(C)O[C@@H]12]

A compound of formula (XXIII′), or a pharmaceutically acceptable salt thereof, is reacted with a P(R²)₃ reagent and an azodicarboxylateto produce a compound of formula (XXV), or a pharmaceutically acceptable salt thereof:

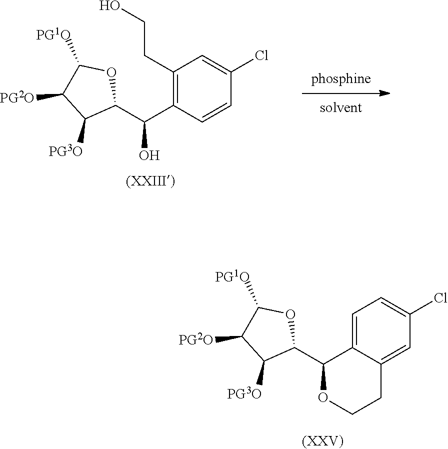

wherein PG¹, PG², and PG³ are as defined with respect to formula (I).

In certain embodiments, PG¹, PG², and PG³ of the compound of formula (XXIII′) and of formula (XXV) are each, independently, a hydroxyl protecting group; or PG² and PG³ together with the oxygen atoms to which they are attached form a 1,2-dihydroxyl protecting group.

In certain embodiments, a compound of formula (XXV) is represented by a compound of formula (IIa) or a compound of formula (IIIa) or a compound of formula (IVa).

In certain embodiments, the compound of formula (XXV) is compound 50 shown below:

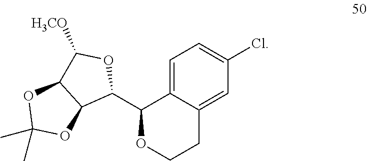 [SMILES: CO[C@@H]1O[C@H]([C@@H]2OCCc3cc(Cl)ccc32)[C@H]2OC(C)(C)O[C@@H]12]

A compound of formula (XXV), or a pharmaceutically acceptable salt thereof, is reacted with acid to produce a compound of formula (XXV′), or a pharmaceutically acceptable salt thereof:

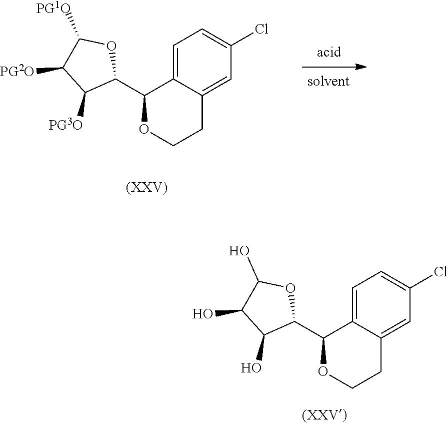

wherein PG¹, PG², and PG³ are as defined with respect to formula (I).

In certain embodiments, PG¹, PG², and PG³ of the compound of formula (XXV) are each, independently, a hydroxyl protecting group; or PG² and PG³ together with the oxygen atoms to which they are attached form a 1,2-dihydroxyl protecting group.

In certain embodiments, a compound of formula (XXV′) is represented by a compound of formula (IVa) or a compound of formula (IVb). In certain embodiments, the compound of formula (XXV′) is compound 60 shown below:

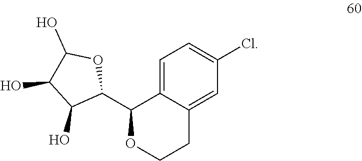

A compound of formula (XXV′), or a pharmaceutically acceptable salt thereof, is reacted with compound (Vb) to produce a compound of formula (VIa-1), or a pharmaceutically acceptable salt thereof:

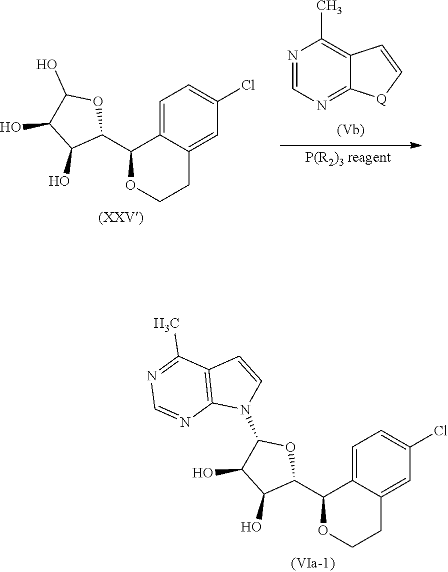

wherein Q is N—H or N⁻K⁺.

In certain embodiments, Q of the compound of formula (Vb) is N⁻K⁺. In certain embodiments, the compound of formula (Vb) is compound 70 shown below:

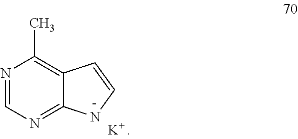

A compound of formula (VIa-1), or a pharmaceutically acceptable salt thereof, is reacted with hydrochloric acid to produce a compound of formula (VIa-3), or a pharmaceutically acceptable salt thereof:

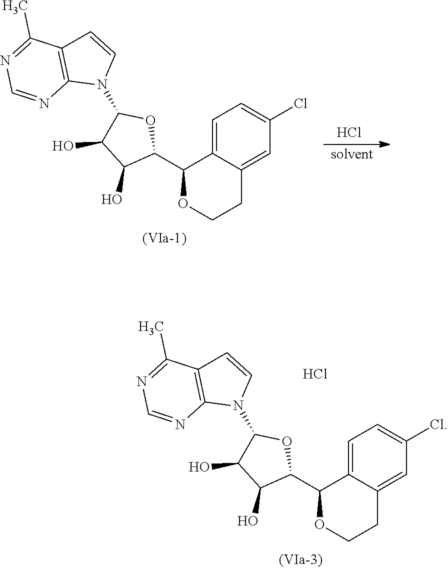 [SMILES: Cc1ncnc2c1ccn2[C@@H]1O[C@H]([C@@H]2OCCc3cc(Cl)ccc32)[C@@H](O)[C@H]1O ; Cl ; Cc1ncnc2c1ccn2[C@@H]1O[C@H]([C@@H]2OCCc3cc(Cl)ccc32)[C@@H](O)[C@H]1O ; Cl]

The following Examples are provided to illustrate aspects of the invention and are not intended to be limiting.

### EXAMPLES

### Example 1—Synthesis A of Compound (VIa-1) and Compound (VIa-3)

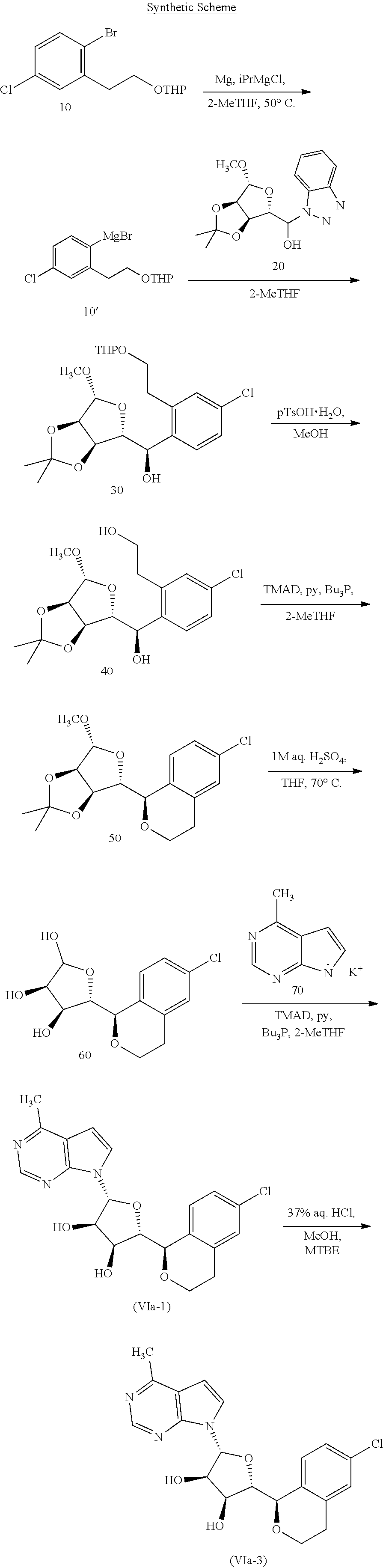

### Synthesis of (1H-benzo[d][1,2,3]triazol-1-yl)((3aR,4S,6R,6aR)-6-methoxy-2,2-dimethyltetra-hydrofuro[3,4-d][1,3]dioxol-4-yl)methanol (Compound 20)

To a solution of crude (3aR,4S,6R,6aR)-6-methoxy-2,2-dimethyltetrahydrofuro [3,4-d][1,3]dioxole-4-carbaldehyde (1.3 g, 6.4 mmol, 1 eq) in MTBE (3 mL, 2.3 mL/g) was added benzotriazole (0.75 g, 6.3 mmol, 0.95 eq) and the resulting solution stirred overnight at ambient temperature to afford a thin, white suspension. Heptanes (9 mL, 3 v/v) were added dropwise, and the suspension stirred at ambient temperature for 1 hour to precipitate additional material prior to isolating the solids by filtration. The filter cake was washed with heptanes (3×1CV) and the solids dried at 50° C. under atmospheric pressure to afford (1H-benzo[d][1,2,3]triazol-1-yl)((3aR,4S,6R,6aR)-6-methoxy-2,2-dimethyltetra-hydrofuro[3,4-d][1,3]dioxol-4-yl)methanol (compound 20, 1.4 g, 70%) as white solids.

### Synthesis of (4-chloro-2-(2-((tetrahydro-2H-pyran-2-yl)oxy)ethyl)phenyl) magnesium bromide (Compound 10′)

To an oven-dried, 2-L four-neck flask equipped with overhead stirrer, thermocouple, and addition funnel, Mg turnings (9.98 g, 411 mmol, 1.05 eq) were suspended in dry 2-MeTHF (190 mL, 1.5 mL/g) under N₂ and the suspension warmed to 50° C. iPrMgCl (9.8 mL, 19.6 mmol, 0.05 eq; 2M in THF) was added via syringe followed by 40 mL (˜10 vol %) of a solution of 2-(2-bromo-5-chlorophenethoxy)tetrahydro-2H-pyran (compound 10, 125 g, 391 mmol, 1 eq) in dry 2-MeTHF (200 mL, 1.6 mL/g; −400 mL total) to initiate Grignard formation. After stirring 10 minutes, an exotherm (e.g., 50 to 56° C.) was observed, indicating the reaction had begun. Remaining compound 10 was charged dropwise at a rate sufficient to maintain an internal temperature<75° C. (Note: External heating was discontinued once the batch temperature reached 65° C.) After completion of the addition, the batch was stirred at 50° C. for 1 hour. HPLC assay of an aliquot quenched with MeOH demonstrated all compound 10 had been consumed.

### Synthesis of (1R)-(4-chloro-2-(2-((tetrahydro-2H-pyran-2-yl)oxy)ethyl)phenyl) ((3aR,4R,6R,6aR)-6-methoxy-2,2-dimethyltetrahydrofuro[3,4-d][1,3]dioxol-4-yl)methanol (Compound 30)

To the four-neck flask containing 1′ (391 mmol, 2.5 eq; typically 0.83-0.86M), (1H-benzo[d][1,2,3]triazol-1-yl)((3aR,4S,6R,6aR)-6-methoxy-2,2-dimethyltetrahydrofuro [3,4-d][1,3]dioxol-4-yl)methanol (compound 20, 50 g, 156 mmol, 1 eq) in 2-MeTHF (500 mL, 10 mL/g) was charged dropwise at ambient temperature under N₂. (Note: use of an ambient temperature water bath as a heat sink is advised during the addition.) The reaction was monitored by HPLC assay of aliquots quenched with MeOH until a constant ratio of product diastereomers to quenched 1′ was observed (˜2.5 hours, demonstrating a ˜4:1 dr) The batch was quenched with 10 wt % aqueous citric acid (1×500 mL, 0.5 v/v) and the layers separated. The organic phase was washed with 4M aqueous NaOH (1×500 mL, 0.5 v/v) then concentrated under reduced pressure to afford a clear, orange-yellow oil that was used directly without further purification.

### Synthesis of 2-(5-chloro-2-((R)-hydroxy((3aR,4R,6R,6aR)-6-methoxy-2,2-dimethyltetra-hydrofuro[3,4-d][1,3]dioxol-4-yl)methyl)phenyl)ethan-1-ol (Compound 40)

The residue containing crude compound 30 (˜150 g) was diluted with MeOH (375 mL, 2.5 mL/g) and pTsOH·H₂O (3.86 g, 20.3 mmol, 0.05 eq to 1′, 0.13 eq to 2) charged at ambient temperature and atmosphere. The batch was stirred at ambient temperature for 1.5 hours, at which time HPLC assay demonstrated the reaction was complete. The reaction was quenched with 1M aqueous NaOH (375 mL, 1 v/v) and extracted with MTBE (3×250 mL, 0.67 v/v). The combined organic layers were concentrated under reduced pressure followed by azeotropic drying with 2-MeTHF (3×volume) to afford crude compound 40 as a clear, orange-yellow syrup that was used directly without further purification.

### Synthesis of (R)-6-chloro-1-((3aR,4R,6R,6aR)-6-methoxy-2,2-dimethyltetra-hydrofuro[3,4-d][1,3]dioxol-4-yl)isochromane (Compound 50)

To a solution of crude compound 40 (˜130 g, 156 mmol, 1 eq) in 2-MeTHF (910 mL, 7 mL/g) in a 2-L three-neck flask equipped with overhead stirrer and thermocouple were charged sequentially pyridine (12.6 mL, 12.33 g, 156 mmol, 1.0 eq), TMAD (67.2 g, 390 mmol, 2.5 eq), and Bu₃P (63.1 g, 312 mmol, 2.0 eq) under N₂. The resulting thin slurry was stirred at ambient temperature for 16 hours, at which time HPLC assay demonstrated the reaction was complete. The batch was quenched with 1M aqueous HCl (900 mL, 1 v/v) and stirred for 1 hour. The layers were separated, and the aqueous phase extracted with 2-MeTHF (1×500 mL, 0.5 v/v). The combined organic layers were concentrated under reduced pressure to afford 207 g of orange oil. The crude product was filtered through a pad of silica gel (621 g, 3 g/g) eluting with heptanes (1×1CV), 5% MTBE in heptanes (1×1CV), then 10% MTBE in heptanes (1×1CV) and the filtrate concentrated under reduced pressure to afford 73 g of faintly yellow oil. This residue was diluted with 9:1 MeOH/H₂O (146 mL, 2 mL/g) at ambient temperature for 2 hours, then cooled in an ice bath for 4 hours. Solids were isolated by filtration, washed with 3:2 MeOH/H₂O (2×150 mL), and dried at 50° C. under atmospheric pressure to afford compound 50 (34.16 g, 64% yield, 92% purity, 21:1 dr) as white, crystalline solids.

### Synthesis of (3R,4S,5S)-5-((R)-6-chloroisochroman-1-yl)tetrahydrofuran-2,3,4-triol (Compound 60)

A solution of compound 50 (3.4 g, 10 mmol, 1 eq) in THF (40 mL, 4 v/v) and 1M aqueous H₂SO₄ (10 mL, 10 mmol, 1.0 eq) was stirred at 70° C. for 20 hours. The reaction was monitored by HPLC and LC-MS assays and when deemed complete, basified to pH 8 with solid NaHCO₃ (1.7 g, 20 mmol) and the layers separated. The aqueous phase was extracted with 2-MeTHF (2×20 mL, 2 v/v). The combined organic layers were concentrated under reduced pressure followed by azeotropic drying with MeCN (3×volume) to afford crude compound 60 as a clear syrup. This residue was diluted with 9:1 Heptane/EA (40 mL) at ambient temperature and stirred overnight. Solids were isolated by filtration, washed with 9:1 Heptane/EA (2×20 mL), and dried at 50° C. under atmospheric pressure to afford compound 60 (2.1 g, 75%, 92% purity) as white solids. LC-MS calc. for C₁₃H₁₅ClO₅ [M−H]⁻: m/z=285.06/287.06; found: 285.06/287.10.

### Synthesis of potassium 4-methylpyrrolo[2,3-d]pyrimidin-7-ide (Compound 70)

To an oven-dried, 100 mL round bottom flask containing 4-methyl-7H-pyrrolo [2,3-d]pyrimidine (1.33 g, 10 mmol, 1 eq) and THF (20 mL, 15 mL/g) under N₂ was charged KOtBu (1.12 g, 10 mmol, 1.0 eq) in portions. The reaction mixture was stirred at ambient temperature for 2 hours, then volatiles removed under reduced pressure. The residue was slurried in MTBE (20 mL, 15 mL/g) overnight, then the solids isolated by filtration, washed with MTBE (2×10 mL), and the solids dried at 50° C. under atmospheric pressure to give compound 70 (1.6 g, 94%) as off-white solids.

### Synthesis of (2S,3S,4R,5R)-2-((R)-6-chloroisochroman-1-yl)-5-(4-methyl-7H-pyrrolo[2,3-d]pyrimidin-7-yl)tetrahydrofuran-3,4-diol (Compound VIa-1)

To an oven-dried, 100 mL round bottom flask containing a solution of compound 60 (715 mg, 2.5 mmol, 1 eq) in MeCN (25 ml, 35 mL/g) were charged successively TMAD (646 mg, 3.75 mmol, 1.5 eq) and Bu₃P (1.2 mL, 5 mmol, 2.0 eq) at ambient temperature under N₂. After stirring for 0.5 hours, a solution of 7 (855 mg, 5.0 mmol, 2.0 eq) in anhydrous DMF (3 mL, 3.5 mL/g) was charged dropwise and the resulting thin slurry stirred overnight at ambient temperature. The reaction was monitored by HPLC assay and when deemed complete, quenched by slow addition of water (30 ml, 1 v/v) and diluted with EtOAc (30 mL, 0.5 v/v) and the layers separated. The organic phase was washed with brine (1×20 mL, 0.67 v/v) and dried over Na₂SO₄. Insolubles were removed by filtration and the filtrate concentrated under reduced pressure to afford a brown oil containing crude compound VIa-1, which was directly without further purification. LC-MS calc. for C₂₀H₂₀ClN₃O₄[M+H]⁺: m/z=402.11/404.11; found: 401.96/403.88.

### Synthesis of (2S,3S,4R,5R)-2-((R)-6-chloroisochroman-1-yl)-5-(4-methyl-7H-pyrrolo[2,3-d]pyrimidin-7-yl)tetrahydrofuran-3,4-diol hydrochloride (Compound VIa-3)

To an oven-dried, 100 mL round bottom flask containing a solution of crude compound VIa-1 (3 g) in MeOH (10 ml, 3.3 mL/g) was charged 37 wt % aqueous HCl (1 mL, 0.1 v/v) dropwise. After stirring 20 minutes at ambient temperature, MTBE (40 ml, 3.6 v/v) was added to precipitate the salt. The resulting slurry was cooled in an ice bath with stirring for 1 hour. Solids were isolated by filtration, the wet cake washed with MTBE (3×1CV) and dried in vacuo at 35° C. to afford compound VIa-3 as white solids.

### Example 2—Synthesis B of Compound (VIa-3)

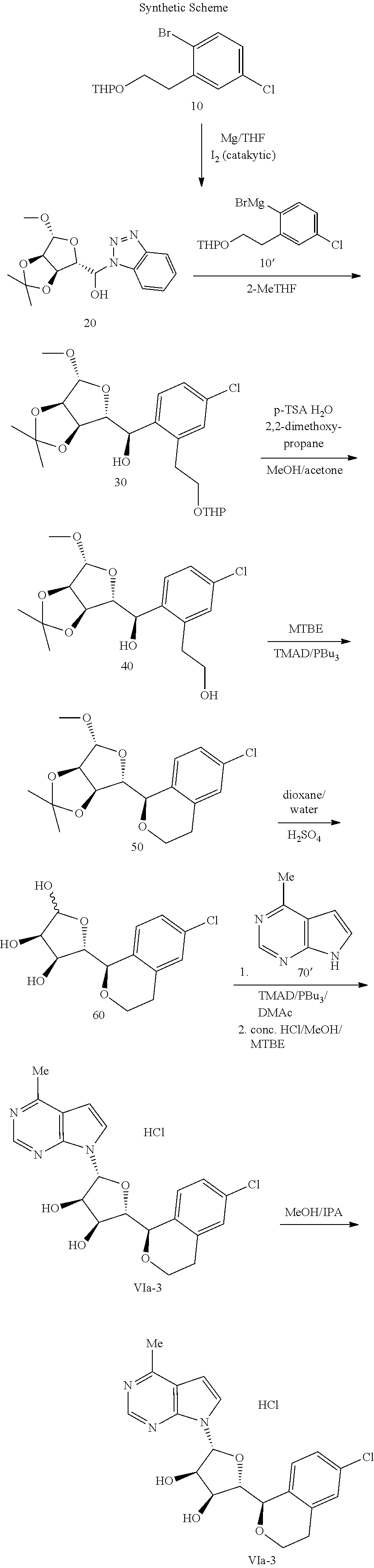

In the step that converts compound 60 to compound VIa-3, NMP may be used as the solvent instead of MTBE.

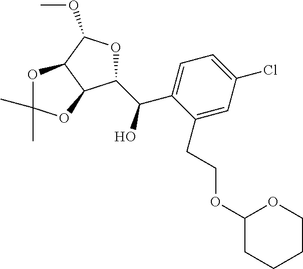 [SMILES: CO[C@@H]1O[C@H]([C@H](O)c2ccc(Cl)cc2CCOC2CCCCO2)[C@H]2OC(C)(C)O[C@@H]12]

### Preparation of the Grignard Reagent

To a clean 100-L reactor charged with Mg turnings (576.8 g, 23.80 mol) under nitrogen was added a solution of 2-(2-bromo-5-chlorophenethoxy)tetrahydro-2H-pyran (compound 10, 360.5 g, 1.13 mol) in tetrahydrofuran (THF, 3.6 L) at 15-25° C. Iodine (4.58 g, 0.036 mol) was charged to the reactor. The resultant mixture was stirred at 30-60° C. [Note: The internal temperature rose to 42° C. upon the addition of Iodine. The initiation of the formation of compound 10′ was confirmed by observing a color change from light yellow to green. The initiation was also indicated by the HPLC. A solution of 2-(2-bromo-5-chlorophenethoxy)tetrahydro-2H-pyran (compound 10, 6343 g, 19.84 mol) in 2-methyl-tetrahydrofuran (2-MeTHF, 11.6 L) was charged to the 100-L reactor while keeping the internal temperature at 20-40° C. The mixture was stirred at 20-40° C. no less than 2 hours before cooled down to 0-10° C.

### Grignard Addition to the Aldehyde Adduct

A solution of (S)-(1H-benzo[d][1,2,3]triazol-1-yl)((3aR,4S,6R,6aR)-6-methoxy-2,2-dimethyltetrahydrofuro[3,4-d][1,3]dioxol-4-yl)methanol (compound 20, 2900 g, 9.02 mol) in 2-MeTHF (11.6 L) was charged to the 100-L reactor with the formed Grignard reagent at the speed of maintaining the batch temperature at 0-20° C. The resultant mixture was then warmed to 20-30° C. and stirred at that temperature for no less than 4 hours. After 12 hours, the reaction mixture was cooled to 0-10° C. A solution of ammonium chloride (NH₄Cl, 2896 g, 54.14 mol) in water (16.5 L) was added to the reaction mixture while maintaining the internal temperature below 30° C. After phase split, the aqueous phase was extracted with 2-MeTHF (11.6 L). The combined organic phases were washed with 75 wt % of a solution of sodium hydroxide (NaOH, 776 g, 19.40 mol) in water (18.6 L) first followed by a second wash with the remaining aqueous NaOH solution. The resultant organic phase was subsequently washed with a solution of sodium chloride (NaCl, 580 g, 9.92 mol) in water (5.2 L). The organic phase was collected and distilled to a minimum agitation volume to provide a crude (1R)-(4-chloro-2-(2-((tetrahydro-2H-pyran-2-yl)oxy)ethyl) phenyl)-((3aR,4R,6R,6aR)-6-methoxy-2,2-dimethyltetrahydrofuro[3,4-d][1,3]dioxol-4-yl) methanol (crude compound 30) (6438 g, 14.54 mol, 162% yield; theoretical yield, 3997 g) as a brown liquid, which was used directly in next reaction without further purification. A small amount of crude compound 30 was purified by column chromatography as a colorless oil.

¹H NMR (400 Hz, DMSO-d₆): δ 7.41 (d, J=8.4 Hz, 1H), 7.31 (d, J=2.4 Hz, 1H), 7.29 (dd, J=8.4, 2.4 Hz, 1H), 5.48 (d, J=6.0 Hz, 1H), 4.98 (d, J=6.1 Hz, 1H), 4.80 (s, 1H), 4.64 (d, J=6.0 Hz, 1H), 4.64-4.54 (m, 2H), 4.35 (d, J=9.3 Hz, 1H), 3.88-3.74 (m, 1H), 3.68 (ddd, J=11.2, 8.3, 5.6 Hz, 1H), 3.58 (tdd, J=10.0, 7.9, 6.3 Hz, 1H), 3.40 (dt, J=10.0, 4.4 Hz, 1H), 3.32 (s, 3H), 3.00 (dt, J=14.1, 7.0 Hz, 1H), 2.93-2.85 (m, 1H), 1.79-1.66 (m, 1H), 1.61 (tq, J=8.6, 2.7 Hz, 1H), 1.54-1.42 (m, 4H), 1.40 (s, 3H), 1.29 (s, 3H) ppm; ¹³C NMR (100 Hz, DMSO-d₆): δ 140.39, 140.36, 139.45, 131.95, 129.84, 126.35, 118.83, 109.68, 98.49, 88.63, 85.06, 82.40, 69.02, 67.31, 61.83, 55.32, 32.10, 30.68, 26.77, 25.49, 25.17, 19.62 ppm.

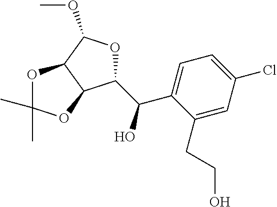 [SMILES: CO[C@@H]1O[C@H]([C@H](O)c2ccc(Cl)cc2CCO)[C@H]2OC(C)(C)O[C@@H]12]

In a clean 100-L reactor under nitrogen was placed crude compound 30 (6438 g, 14.54 mol; theoretical yield for Step 1 is 3997 g of compound 3, 9.02 mol) in methanol (MeOH, 16.0 L) and acetone (26.0 L) at 15-25° C. To the solution was charged 2,2-dimethoxypropane (1409.7 g, 13.53 mol) and p-toluenesulfonic acid monohydrate (p-TSA-H₂O, 512 g, 2.69 mol) in sequence at 15-30° C. The reaction mixture was stirred at 15-30° C. for no less than 1 hour. Upon the completion of the reaction indicated by HPLC (Criteria: <3% of compound 30 vs compound 40), a solution of sodium bicarbonate (NaHCO₃, 600 g, 7.14 mol) in water (11.2 L) was added to the reaction mixture while maintaining the internal temperature below 30° C. The mixture was distilled at <50° C. under vacuum to about 24 L (˜6 vol.). Dichloromethane (DCM, 12.0 L) was added to the resultant mixture. The organic phase was separated, washed with water (12.0 L) and distilled to a minimum agitation volume to provide a crude 2-(5-chloro-2-((R)-hydroxy((3aR,4R,6R,6aR)-6-methoxy-2,2-dimethyltetrahydrofuro[3,4-d][1,3]dioxol-4-yl)methyl)phenyl)ethan-1-ol (crude compound 40) (5576 g, 15.54 mol, 172% yield; theoretical yield 3237.9 g, 9.02 mol) as a brown liquid, which was used directly in next reaction without further purification. A small amount of crude compound 40 was purified by column chromatography to provide compound 40 as a colorless oil.

¹H NMR (400 Hz, DMSO-d₆): δ 7.44 (d, J=8.8 Hz, 1H), 7.29-7.22 (m, 2H), 5.44 (d, J=6.0 Hz, 1H), 4.98 (dd, J=6.0, 0.9 Hz, 1H), 4.80 (s, 1H), 4.77 (t, J=5.0 Hz, 1H), 4.63 (d, J=6.0 Hz, 1H), 4.57 (dd, J=9.6, 6.0 Hz, 1H), 4.35 (dd, J=9.5, 0.9 Hz, 1H), 3.61 (dt, J=11.7, 7.0 Hz, 2H), 3.32 (s, 3H), 2.93-2.86 (m, 1H), 2.77 (dt, J=13.9, 7.1 Hz, 1H), 1.40 (s, 3H), 1.29 (s, 3H) ppm; ¹³C NMR (100 Hz, DMSO-d₆): δ 141.02, 139.45, 131.91, 129.88, 129.68, 126.19, 111.83, 109.61, 88.52, 85.08, 82.46, 68.69, 61.87, 35.40, 26.78, 25.18 ppm.

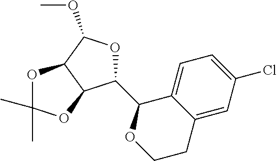 [SMILES: CO[C@@H]1O[C@H]([C@@H]2OCCc3cc(Cl)ccc32)[C@H]2OC(C)(C)O[C@@H]12]

In a clean 100-L reactor under nitrogen was placed crude compound 40 (5576 g, 15.54 mol, theoretical yield 3237.9 g, 9.02 mol) in methyl tert-butyl ether (MTBE, 32.4 L) at 15-30° C. To the solution was charged tetramethylazodicarboxamide (TMAD, 1927 g, 11.19 mol) at 15-30° C. The mixture was stirred at that temperature for 1 hour before cooled to 0-10° C. Tri-n-butylphosphine (P(n-Bu)₃, 2614.7 g, 12.92 mol) was added to the solution while maintaining the internal temperature below 15° C. The resultant reaction mixture was warmed to 15-30° C. and stirred at that temperature range for no less than one hour. Upon the completion of the reaction indicated by HPLC (Criteria: <3% of compound 40 vs compound 50), a solution of NaCl (2590 g, 44.32 mol) in water (23.3 L) was added while maintaining the batch temperature below 30° C. The organic phase was separated and distilled at <50° C. under vacuum to the minimum agitation volume. To the resultant residue was added MeOH (12.6 L) followed by addition of water (7.1 L) slowly. The mixture was stirred at 15-30° C. for 12 hours and then cooled to 0-10° C. After stirred at 0-10° C. for 2 hours, the solid was collected by filtration and was subsequently washed with a mixed solvents of MeOH (3.2 L) and water (3.2 L). The wet cake was dried on the filter for 22 hours to provide (R)-6-chloro-1-((3aR,4R,6R,6aR)-6-methoxy-2,2-dimethyltetrahydrofuro[3,4-d][1,3]dioxol-4-yl)isochromane (compound 50, 1826 g, 5.36 mol; yield for 3 steps 59.4%) as a white solid.

¹H NMR (400 Hz, DMSO-d₆): δ 7.63-7.51 (m, 1H), 7.28-7.25 (m, 2H), 5.06 (s, 1H), 4.94 (dd, J=6.1, 1.5 Hz, 1H), 4.61 (d, J=6.0 Hz, 1H), 4.56-4.46 (m, 1H), 4.17 (dd, J=8.3, 1.6 Hz, 1H), 4.07 (ddd, J=11.2, 5.6, 3.6 Hz, 1H), 3.68 (ddd, J=11.2, 9.5, 4.0 Hz, 1H), 3.36 (s, 3H), 2.90 (ddd, J=15.7, 9.4, 5.7 Hz, 1H), 2.72 (dt, J=16.7, 3.9 Hz, 1H), 1.38 (s, 3H), 1.26 (s, 3H) ppm; ¹³C NMR (100 Hz, DMSO-d₆): δ 137.18, 135.18, 128.81, 128.28, 126.33, 112.11, 109.99, 89.48, 84.58, 81.31, 74.78, 63.05, 55.66, 28.68, 26.98, 25.35 ppm.

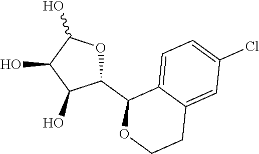

In a clean reactor under nitrogen was placed compound 50 (3037 g, 8.91 mol) in 1,4-dioxane (9.1 L) at 15-25° C. To the solution was added a solution of sulfuric acid (H₂SO₄, 305.5 g, 3.12 mol) in water (30.4 L). The reaction mixture was heated to 60-70° C. and stirred at that temperature for no less than 14 hours. Upon the completion of the reaction indicated by HPLC, the batch was cooled to 20-40° C. Ethyl acetate (EtOAc, 36.4 L) was charged to the reaction mixture followed by charging solid NaHCO₃ (916.9 g, 10.92 mol) in portions to adjust the pH to 8-9 while the internal temperature below 40° C. To the mixture was added NaCl (4560 g, 77.95 mol), and the resultant organic phase was separated and concentrated to a minimum agitation volume. Toluene (20.0 L) and MTBE (9.1 L) were added to the distillation residue subsequently. After the resultant mixture was stirred at 15-30° C. for 1 hour, n-heptane (2.7 L) was added. The suspension was stirred at 15-30° C. for 2 hours. An extra amount of n-heptane (6.4 L) was added, and the mixture was stirred at 15-30° C. for another 2 hours. The resultant solid was collected by filtration and the wet cake was washed with a mixed solvents of toluene (3.0 L) and n-heptane (6.1 L). The filter cake was transferred to trays and dried at <35° C. in an oven under vacuum to provide (3R,4S,5S)-5-((R)-6-chloroisochroman-1-yl)tetrahydrofuran-2,3,4-triol (compound 60, 2302 g, 8.03 mol; yield 90.1%) as an off-white solid. Compound 60 was isolated a mixture of isomers (˜1:1).

¹H NMR (400 Hz, DMSO-d₆): δ 7.46 (d, J=8.2 Hz, 0.5H), 7.33 (d, J=8.2 Hz, 0.5H), 7.25-7.19 (m, 2H), 6.33 (d, J=5.6 Hz, 0.5H), 5.80 (d, J=7.0 Hz, 0.5H), 5.12 (dd, J=7.0, 4.3 Hz, 0.5H), 5.01 (dd, J=5.6, 2.9 Hz, 0.5H), 4.83 (d, J=5.3 Hz, 0.5H), 4.69 (d, J=4.0 Hz, 0.5H), 4.61 (d, J=6.2 Hz, 0.5H), 4.60 (d, J=5.6 Hz, 0.5H), 4.57 (d, J=5.8 Hz, 0.5H), 4.44 (d, J=8.1 Hz, 0.5H), 4.32 (d, J=2.8 Hz, 0.5H), 4.31 (d, J=2.8 Hz, 0.5H), 4.13-4.00 (m, 1.5H), 3.94 (dd, J=6.5, 4.4 Hz, 0.5H), 3.85 (ddd, J=8.1, 6.0, 4.3 Hz, 0.5H), 3.76 (td, J=5.9, 2.8 Hz, 0.5H), 3.72-3.66 (m, 0.5H), 3.60 (td, J=10.8, 3.5 Hz, 0.5H), 2.92-2.80 (m, 1H), 2.78-2.61 (m, 1H) ppm; ¹³C NMR (100 Hz, DMSO-d₆): δ (Set 1) 137.00, 135.14, 131.40, 128.98, 128.55, 125.91, 102.11, 84.99, 76.37, 76.00, 72.04, 62.46, 28.85; (Set 2) 137.44, 134.13, 131.43, 128.78, 128.21, 126.28, 96.94, 86.68, 75.79, 71.68, 70.18, 63.12, 28.85 ppm; C₁₃H₁₅ClO₅ (Mol. Wt: 286.71), LCMS (EI) m/e 285.06 [M−H]⁻.

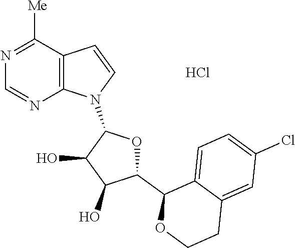 [SMILES: Cc1ncnc2c1ccn2[C@@H]1O[C@H]([C@@H]2OCCc3cc(Cl)ccc32)[C@@H](O)[C@H]1O ; Cl]

In a clean reactor under nitrogen were placed compound 60 (1772 g, 6.18 mol) and 4-methyl-7H-pyrrolo[2,3-d]pyrimidine (compound 70′, 1070 g, 8.04 mol) in N,N-dimethylacetamide (DMAc, 17.7 L) at 15-25° C. To the resultant solution was added anhydrous potassium phosphate (K₃PO₄, 852 g, 4.01 mol). After 30 minutes, TMAD (2022 g, 11.74 mol) was added to the reactor at 15-25° C. followed by adding P(n-Bu)₃ (2381 g, 11.77 mol) in 5 hours while maintaining the internal temperature at 20-35° C. The reaction mixture was stirred at 20-35° C. for no less than 16 hours. Upon the completion of the reaction indicated by HPLC (Criteria: <3% of compound 60 vs compound VIa-3), the batch was filtered, and the filtrate was collected. The reactor was rinsed with EtOAc (26.6 L) and the rinse mixture was used to wash the resultant filter cake. The filtrates were combined and cooled to 0-10° C. To the solution was added a solution of NaCl (436 g) in water (43.6 L) while maintaining the batch temperature below 30° C. The mixture was stirred for 30 minutes, and the layers were separated. The aqueous layer was extracted with EtOAc (2×26.6 L). All the organic phases were combined and washed by a solution of NaCl (145 g) in water (14.5 L) twice. The separated organic phase was distilled at <55° C. under vacuum to the minimum agitation volume. To the distillation residue was added MeOH (10.3 L) and subsequently a solution of concentrated hydrochloric acid (HCl, 12 M, 1.71 L, 20.5 mol) in MeOH (6.8 L) while maintaining the batch temperature below 30° C. After stirred at 15-30° C. for 30 minutes, MTBE (68.2 L) was charged. The mixture was heated to 35-45° C. for 1 hour before cooled to 0-10° C. After stirred at 0-10° C. for 1 hour, the solid was collected by filtration.

The wet cake was washed with a mixed solvents of MeOH (4.5 L) and MTBE (18.2 L). The resultant cake was dried on the filter under vacuum for 16 hours, then was transferred to a clean reactor. To the reactor was charged isopropanol (IPA, 11.8 L) and dichloromethane (DCM, 2.0 L). The suspension was heated to 35-45° C. and stirred at that temperature range for 1 hour. The hot slurry was then cooled to 10-20° C. After stirred at 10-20° C. for 1 hour, The solid was collected by filtration and washed with a mixed solvents of IPA (3.4 L) and DCM (0.56 L). The resultant cake was dried on the filter under vacuum for 16 hours to provide (2S,3S,4R,5R)-2-((R)-6-chloroisochroman-1-yl)-5-(4-methyl-7H-pyrrolo [2,3-d]pyrimidin-7-yl)tetrahydrofuran-3,4-diol hydrochloride (crude compound VIa-3, 1355 g, 3.09 mol; yield, 50%) as a white to off-white solid.

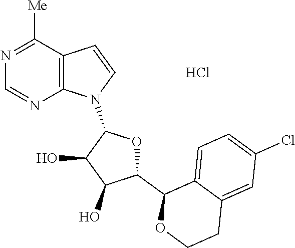 [SMILES: Cc1ncnc2c1ccn2[C@@H]1O[C@H]([C@@H]2OCCc3cc(Cl)ccc32)[C@@H](O)[C@H]1O ; Cl]

In a clean container was placed crude compound VIa-3 (1742 g, 3.97 mol) in MeOH (7.8 L) at 15-25° C. The solution was filtered through an in-line filter to a clean reactor under nitrogen. The container was rinsed with MeOH (0.9 L) and the rinse solution was subsequently filtered through the same in-line filter to the reactor. The solution was distilled at atmosphere pressure while adding polish filtered 2-propanol (IPA, 12.2 L) in portions. The distillation was continued until the solid precipitation was observed. The mixture was then cooled to 15-25° C. and stirred at that temperature for 2 hours. The solid was collected by filtration, washed with a polish filtered mixed solvents of DCM (0.57 L) and IPA (3.5 L). After washed by a polish filtered n-heptane (5.2 L), the cake was dried on the filter for 3 hours and was further dried in drying trays at <60° C. under vacuum. The dried solid was ground and passed through a 100 mesh sieve. The resultant solid was dried in drying trays at <60° C. under vacuum to provide (2S,3S,4R,5R)-2-((R)-6-chloroisochroman-1-yl)-5-(4-methyl-7H-pyrrolo[2,3-d]pyrimidin-7-yl)tetrahydrofuran-3,4-diol hydrochloride (compound VIa-3, 1498 g, 86%) as a white to off-white solid.

1. A process for preparing a compound of formula (II), or a pharmaceutically acceptable salt thereof, comprising combining a compound of formula (I), or a pharmaceutically acceptable salt thereof, with a P(R²)₃ reagent and an azodicarboxylate or azodicarboxamide, in the presence of an organic solvent: 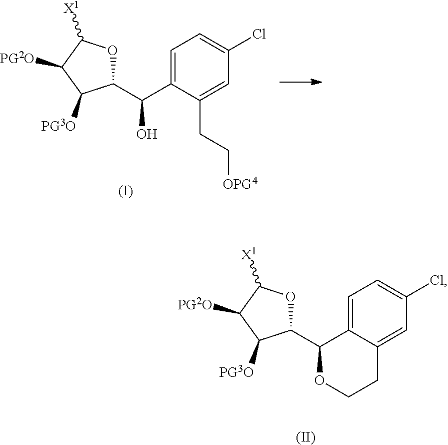
wherein
X¹ is OH or OPG¹;
PG¹ is a hydroxyl protecting group;
PG², PG³ and PG⁴ are each independently H or a hydroxyl protecting group;
or PG² and PG³ together with the oxygen atoms to which they are attached form a 1,2-dihydroxyl protecting group; and
each R² is independently C₁-C₆alkyl or aryl.

2. The process of claim 1, wherein the compound of formula (I) is a compound of formula (Ia) or a pharmaceutically acceptable salt thereof and the compound of formula (II) is a compound of formula (IIa) or a pharmaceutically acceptable salt thereof:

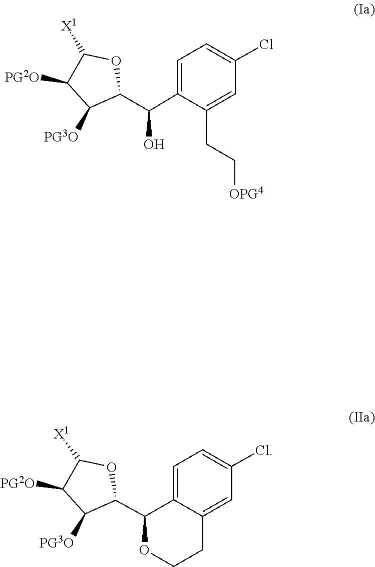

3. The process of claim 1, wherein PG² and PG³ together with the atoms to which they are attached, form a 1,2-dihydroxyl protecting group, wherein the 1,2-dihydroxyl protecting group is an acetonide moiety.

4. (canceled)

5. The process of claim 2, wherein the compound of formula (Ia) is formula (Ia-1) or a pharmaceutically acceptable salt thereof:

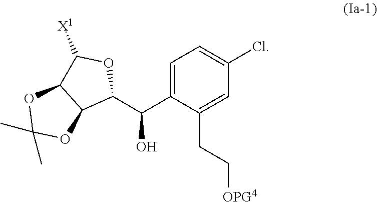

6. The process of claim 2, wherein the compound of formula (IIa) is the compound of formula (IIa-1) or a pharmaceutically acceptable salt thereof:

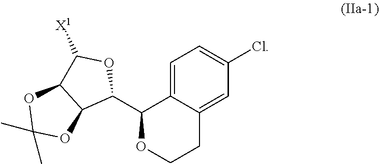

7. The process of claim 1, wherein X¹ is OH or OPG¹.

8. (canceled)

9. The process of claim 1, wherein the compound of formula (I) is combined with the P(R²)₃ reagent wherein R² is C₁-C₆ alkyl and the phosphine is trimethylphosphine, triethylphosphine, tri-n-propyl-phosphine or tri-n-butyl-phosphine or wherein R² is aryl the phosphine is triphenylphosphine, (p-dimethylaminophenyl)diphenylphosphine or diphenyl-2-pyridylphosphine.

10. (canceled)

11. (canceled)

12. The process of claim 1, wherein the azodicarboxylate or azodicarboxamide or derivative thereof is diisopropyl-azodicarboxylate (DIAD), tetramethyl azodicarboxamide (TMAD) or diethyl-azodicarboxylate (DEAD).

13. (canceled)

14. (canceled)

15. The process of claim 1, wherein the organic solvent is diethyl ether, t-butyl methyl ether, dichloromethane or tetrahydrofuran, or a combination thereof.

16. The process of claim 1, wherein the compound of formula (II) is a compound of formula (III) or a pharmaceutically acceptable salt thereof, wherein PG¹ is an acid labile hydroxyl protecting group and wherein the compound of formula (III) or the pharmaceutically acceptable salt thereof is treated with an acid for a time and under conditions sufficient to provide a compound of formula (IV) or a pharmaceutically acceptable salt thereof:

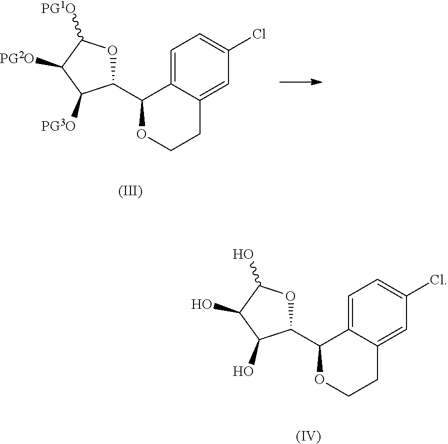

17. (canceled)

18. The process of claim 17, wherein the compound of formula (IIIa) is the compound of formula (IIIa-1) or a pharmaceutically acceptable salt thereof:

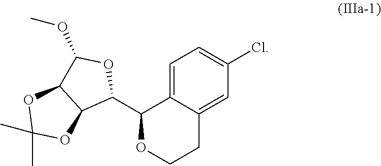 [SMILES: CO[C@@H]1O[C@H]([C@@H]2OCCc3cc(Cl)ccc32)[C@H]2OC(C)(C)O[C@@H]12]

19. (canceled)

20. The process of claim 16, wherein the acid is HCl, H₃PO₄, or H₂SO₄.

21. (canceled)

22. (canceled)

23. (canceled)

24. The process of claim 16, further comprising reacting the compound of formula (IV) or a pharmaceutically acceptable salt thereof with a compound of formula (V) in the presence of a P(R²)₃ reagent and an azodicarboxylate or azodicarboxamide, in the presence of an organic solvent, to produce a compound of formula (VI) or a pharmaceutically acceptable salt thereof: 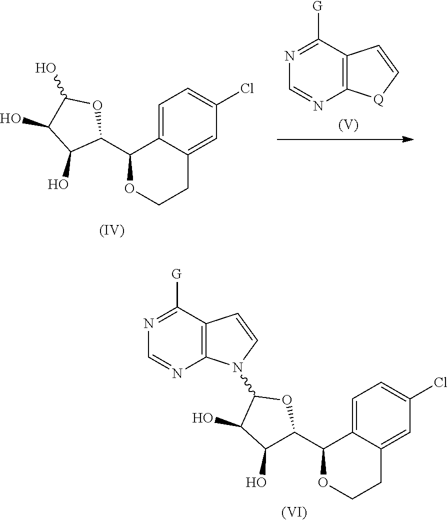
wherein G is a halogen or C₁-C₆alkyl; Q is N—H or N⁻K⁺ and each R² is independently C₁-C₆alkyl or aryl.

25. (canceled)

26. (canceled)

27. (canceled)

28. (canceled)

29. The process of claim 24, wherein the G halogen is —Cl, —Br, or —I or methyl, and wherein R² is C₁-C₆ alkyl or aryl, and the phosphine reagent is trimethylphosphine, triethylphosphine, tri-n-propylphosphine or tri-n-butyl-phosphine, wherein the organic solvent is diethyl ether, t-butyl methyl ether, dichloromethane or tetrahydrofuran, or a combination thereof.

30. (canceled)

31. (canceled)

32. (canceled)

33. (canceled)

34. (canceled)

35. (canceled)

36. (canceled)

37. (canceled)

38. (canceled)

39. (canceled)

40. The process of claim 24, wherein the compound of formula (V) is the compound of formula (Va): 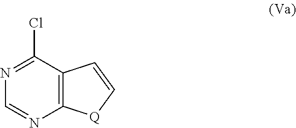
or wherein the compound of formula (V) is the compound of formula (Vb):

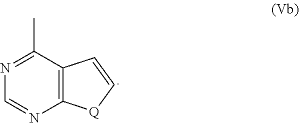

41. (canceled)

42. The process of claim 40, wherein the compound of formula (V) is compound 70:

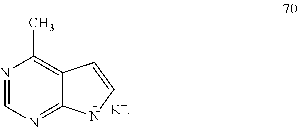

43. The process of claim 24, wherein the compound of formula (VI) is the compound of formula (VIa-2): 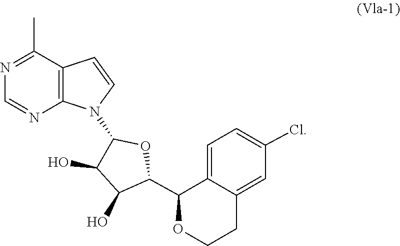 [SMILES: Cc1ncnc2c1ccn2[C@@H]1O[C@H]([C@@H]2OCCc3cc(Cl)ccc32)[C@@H](O)[C@H]1O]
or wherein the compound of formula (VI) is a compound of formula VIa-1):

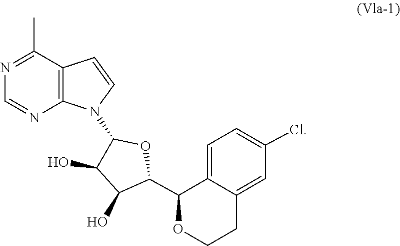 [SMILES: Cc1ncnc2c1ccn2[C@@H]1O[C@H]([C@@H]2OCCc3cc(Cl)ccc32)[C@@H](O)[C@H]1O]

44. (canceled)

45. The process of claim 43, further comprising reacting the compound of formula (VIa-1) with hydrochloric acid to form to form a pharmaceutically acceptable salt of formula (VIa-3):

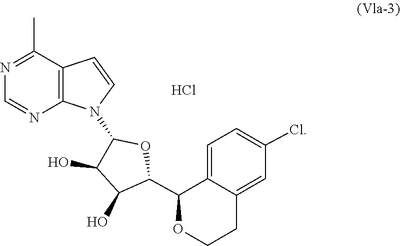 [SMILES: Cc1ncnc2c1ccn2[C@@H]1O[C@H]([C@@H]2OCCc3cc(Cl)ccc32)[C@@H](O)[C@H]1O ; Cl]

46. (canceled)

47. The process according to claim 1 wherein the compound of formula (I) is a compound of formula (XXIII), or a pharmaceutically acceptable salt thereof, wherein the compound of formula (XXIII) is prepared by reacting a compound of formula (XXI′) with a compound of formula (XXII):

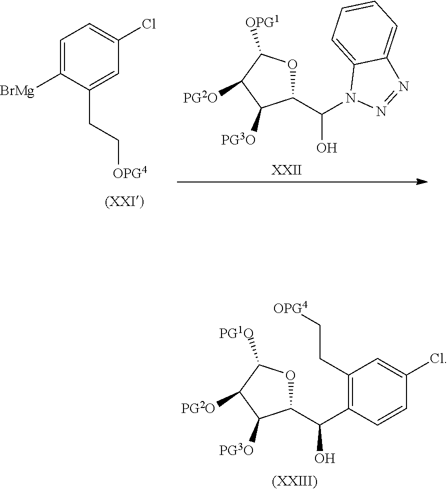

48. The process of claim 47, wherein PG⁴ is a THP moiety and wherein PG² and PG³ together with the oxygen atoms to which they are attached form a 1,2-dihydroxyl protecting group.

49. (canceled)

50. The process of claim 47, wherein the compound of formula (XXII) is compound 20: 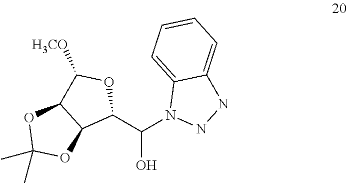 [SMILES: CO[C@@H]1O[C@H](C(O)N2NNc3ccccc32)[C@H]2OC(C)(C)O[C@@H]12]
and wherein the compound of formula (XXIII) is compound 30:

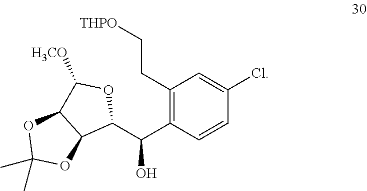

51. (canceled)

52. The process of claim 47, further comprising reacting the compound of formula (XXIII), or a pharmaceutically acceptable salt thereof, with an alcohol to produce a compound of formula (XXIII′), or a pharmaceutically acceptable salt thereof:

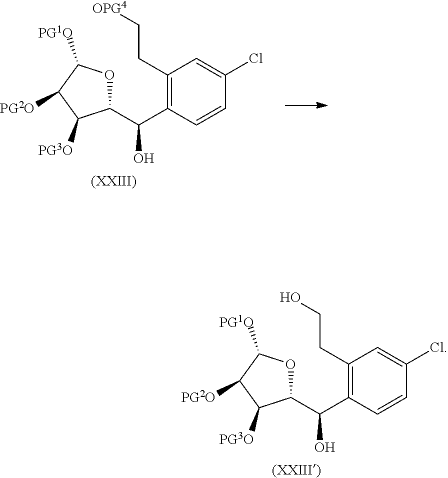

53. The process of claim 52, wherein PG⁴ is a THP moiety and wherein PG² and PG³ together with the oxygen atoms to which they are attached form a 1,2-dihydroxyl protecting group.

54. (canceled)

55. The process of claim 52, wherein the compound of formula (XXIII′) is compound 40:

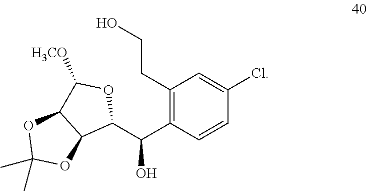 [SMILES: CO[C@@H]1O[C@H]([C@H](O)c2ccc(Cl)cc2CCO)[C@H]2OC(C)(C)O[C@@H]12]

56. The process of any one of claim 52, further comprising reacting the compound of formula (XXIII′), or a pharmaceutically acceptable salt thereof, with a P(R2)3 reagent to produce a compound of formula (XXV), or a pharmaceutically acceptable salt thereof, wherein PG2 and PG3 together with the oxygen atoms to which they are attached form a 1,2-dihydroxyl protecting group:

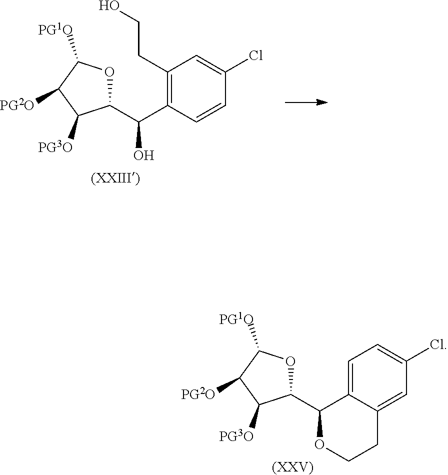

57. (canceled)

58. The process of claim 56, wherein the compound of formula (XXV) is compound 50:

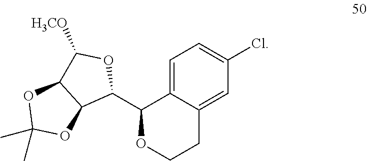 [SMILES: CO[C@@H]1O[C@H]([C@@H]2OCCc3cc(Cl)ccc32)[C@H]2OC(C)(C)O[C@@H]12]

59. The process of claim 56, further comprising reacting the compound of formula (XXV), or a pharmaceutically acceptable salt thereof, with an acid to produce a compound of formula (XXV′), or a pharmaceutically acceptable salt thereof, wherein PG² and PG³ together with the oxygen atoms to which they are attached form a 1,2-dihydroxyl protecting group, wherein the acid is sulfuric acid:

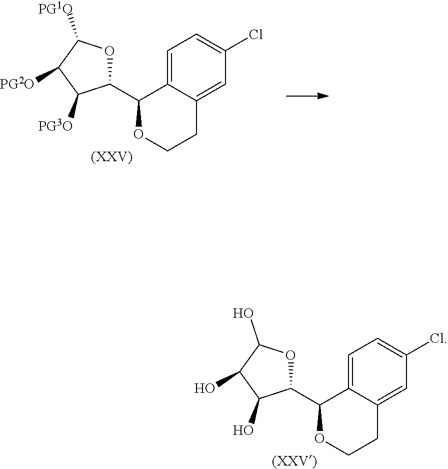

60. (canceled)

61. (canceled)

62. The process of claim 59, wherein the compound of formula (XXV′) is compound 60:

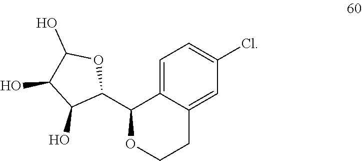

63. The process of claim 62, wherein the compound of formula (XXV′) is a compound of formula (IVa-1) or a compound of formula (IVb-1)

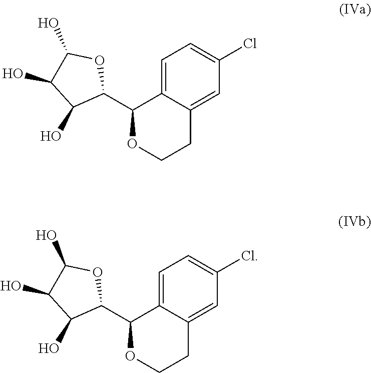 [SMILES: O[C@@H]1[C@H](O)[C@@H]([C@@H]2OCCc3cc(Cl)ccc32)O[C@H]1O ; O[C@@H]1[C@H](O)[C@@H]([C@@H]2OCCc3cc(Cl)ccc32)O[C@@H]1O]

64. The process of claim 24, further comprising converting the compound of formula (IV) to an epoxide of formula (IVaa) by reacting the compound of formula (IV) with a P(R²)₃ reagent, an azodicarboxylate or azodicarboxamide, in the presence of an organic solvent to produce a compound of formula (IVaa): 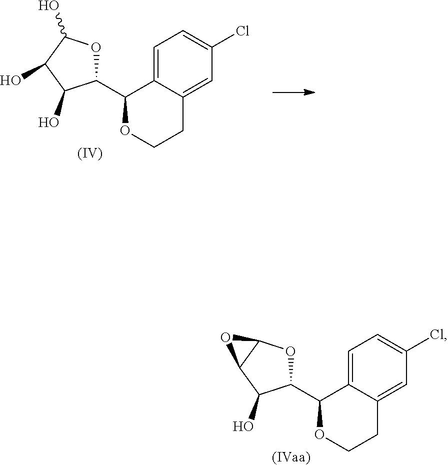
wherein each R² is independently C₁-C₆alkyl or aryl.

65. The process of claim 64, further comprising reacting the compound of formula (IVaa) with a compound of formula (V), or a basic salt thereof, with a P(R²)₃ reagent, an azodicarboxylate or azodicarboxamide, in the presence of an organic solvent to produce a compound of formula (VIa): 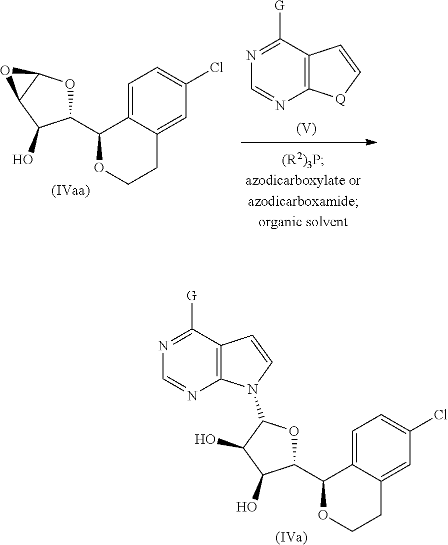
wherein G is a halogen or C₁-C₆alkyl; each R² is independently C₁-C₆alkyl or aryl; and Q is —N—H or N⁻K⁺, wherein the P(R²)₃ reagent is triphenylphosphine or tributylphosphine, and the azodicarboxylate or azodicarboxamide is diethylazodi-carboxylate (DEAD), diisopropylazodicarboxylate (DIAD), or tetramethyl azodicarboxamide (TMAD).

66. (canceled)

67. A compound that is compound 50 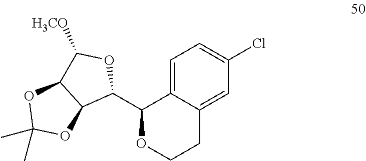 [SMILES: CO[C@@H]1O[C@H]([C@@H]2OCCc3cc(Cl)ccc32)[C@H]2OC(C)(C)O[C@@H]12]
or a pharmaceutically acceptable salt thereof or compound 60 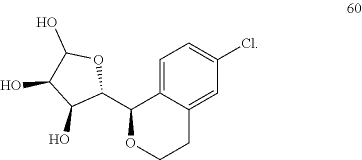
or a pharmaceutically acceptable salt thereof.

68. (canceled)
